(19) United States
(12) Patent Application Publication
(10) Pub. No.: US 2021/0246643 A1
(43) Pub. Date: Aug. 12, 2021

(54) SYSTEMS DEVICES AND METHODS FOR DETECTING AND DIAGNOSING SUBSTANCES

(71) Applicant: Binyamin Yefet SONOVANI, Qatsrin (IL)

(72) Inventor: Binyamin Yefet SONOVANI, Qatsrin (IL)

(21) Appl. No.: 15/734,731

(22) PCT Filed: Jun. 4, 2019
(86) PCT No.: PCT/IL2019/050637

§ 371 Date: Dec. 3, 2020

Related U.S. Application Data
(60) Provisional application No. 62/680,157, filed on Jun. 4, 2018.

(51) Int. Cl. E03D 9/00; G01N 1/02; A61L 2/24; A61L 2/22; H04N 7/18; G06K 9/00

(52) U.S. Cl. CPC E03D 9/005; G01N 1/02; A61L 2/24; A61L 2101/34; H04N 7/18; G06K 9/00624; A61L 2/22

(57) ABSTRACT

In accordance with embodiments, there are provided devices, systems and methods for capturing images of one or more substances in a scene, such as toilets using an illumination module and an imaging module included in a housing wherein the housing is configured to be inserted to devices such as toilet cleaning devices and receive the one or more substances. The imaging module comprising one or more high quality imagers configured to capture one or more images or an image video of the substances and a processor for analyzing the captured images to identify the characteristic and/or type of the substances.

### CROSS-REFERENCE

The present application claims the benefit of U.S. Provisional Application Ser. No. 62/680,157 entitled “SYSTEM DEVICE AND METHODS FOR DETECTING AND DIAGNOSING SUBSTANCES IN TOILETS” filed on Jun. 4, 2018 and PCT application number PCT/IL2019/050637 filed on Jun. 4, 2019 which are both incorporated herein by reference in its entirety.

### TECHNICAL FIELD OF THE INVENTION

The present invention relates generally to the detection and diagnosing of substances such as infectors, drugs, explosive or toxic compounds and more particularly, to a devices systems and methods for detecting and alerting of threats such as infectors explosives drugs or toxic compounds found in a scene, such as a toilet or toilets compartments.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BACKGROUND OF THE INVENTION

The massive terror attacks which the world witnessed in the last decades, starting with the 9/11 attack in the US and later on in the London underground station and terror incidents in France, changed dramatically the security measures taken by governments, municipals and public authorities such as airfields stadiums universities etc. For example, the Department of Homeland Security was established in the US to secure and resilient nation to effectively prevent terrorism. However, terrorist, drug dealers and saboteurs tactics continue to evolve, as they seek sophisticated means of attack, including chemical, biological, radiological, nuclear and explosive weapons, and cyber-attacks.

As a result, there is a significant demand for devices and methods that can rapidly detect chemical-biological-explosive (CBE) threats on-site and allow for immediate responders to mitigate spread, risk, and loss. In general, the key to an effective reconnaissance mission is a unified detection technology that analyzes potential threats in real time, and specifically in areas where the terrorist or the threat may be easily and immediately monitored.

### SUMMARY OF THE INVENTION

According to a first embodiment there is provided a device for collecting and characterizing substances in a toilet and cleaning the toilet, the device comprises: a storage and control module, comprising: at least one container, said at least one container is configured to store one or more cleaning materials of a first type for cleaning or sanitizing said toilet; one or more pumps for moving via one or more pipes said one or more cleaning materials of a first type; at least one diagnostic module configured to receive said substances and diagnose the received substances, the diagnostic module comprises: a housing having a cavity therein wherein the cavity is configured to contain said substances; one or more openings for receiving said substances via one or more pipes; an imaging module comprising one or more imagers for capturing one or more images of the substances; an illumination module comprising one or more illumination sources for illuminating the substances; a sensor module comprising one or more sensors for obtaining sensory data of the substances; at least one processor in communication with said sensor module and imaging module, said at least one processor is configured to receive said sensory data and said captured images and analyze said sensory data and said captured images to identify the characteristics of said substances; and one or more power sources; a treatment module, said treatment module comprising: a housing having a cavity configured and enabled to collect and store flushing tank fluids and said substances of said toilet flowing via one or more openings in said housing; one or more cleaning materials of a second type; a spraying module, comprising one or more spraying devices, wherein said spraying devices are configured and enabled to spray said flushing tank fluids mixed with the cleaning materials of the second type via the one or more spraying devices into to the interior of said toilet bowl; one or more pumps for drawing said flushing tank fluids mixed within the treatment module with a second type of cleaning materials to the spraying module; a rotatable spraying module configured to: receive cleaning materials of a first type from said one or more pipes; rotate perpendicularly in respect to said treatment module length at the said toilet bowl cavity; and spray said cleaning materials of a first type at said toilet bowl cavity; a bridge module connectable to said treatment module and said treatment module, said bridge module is configured and enabled to be hanged on said toilet bowl rim and hold said treatment module away and below the toilet bowl rim at the path of the toilet flushing water;

According to some embodiments, the device comprise a mobile communication device comprising said processor and wireless communication circuitry to couple to the device and communicate with a remote server, the processor comprising instructions to transmit the captured images and the sensory data of the substances to the remote server and receive substances data in response to the captured images and the sensory data from the remote server.

According to some embodiments, the remote server comprises a database and a tangible medium embodying instructions of an algorithm to compare the captured images and sensory data to the database.

According to some embodiments, substance data comprises one or more of an identification of the substances, a classification of the substances among a plurality of classifications, one or more components of the substances, or food categories of the substances.

According to some embodiments, the treatment module comprises a collecting module attached externally to the treatment module, said collecting module is configured to collect said substances and insert them into the treatment module.

According to some embodiments, the collecting module comprises one or more of: mechanical arms; tubules for collecting and sucking the substances; whip type element configured to preform whiplash movement and stick the substances within the toilet to it's end.

According to some embodiments, the device comprising an optical fiber comprising one or more imagers attached to said optical fiber distal end said imagers are configured to capture one or more images of the substances and transmit the one or more images to the said device and/or to an external device.

According to a second embodiment there is provided a device for characterizing substances in a scene, the device comprising: a housing having a cavity therein wherein the cavity is configured to contain said substances; one or more openings for receiving said substances from the scene; an imaging module comprising one or more imagers or video imagers of high resolution for capturing one or more images of the substances; an illumination module comprising one or more illumination sources for illuminating the substances; a sensor module comprising one or more sensors for obtaining sensory data of the substances; a gas supply device for storing and inserting gas into the housing; a mechanical module configured to accordingly insert or take out the substances into or from the device via the one or more openings; a syringe configured to add one or more materials into the housing; a pump for creating vacuum in the device; an air pump configured to suck air from the housing and take out the substances from the housing; a fan for cooling the housing; a fluid injector for inserting fluid into the housing; one or more opening for inserting working tools to sterilely move or take the substances or control other modules in the device; at least one processor in communication with said sensor module and imaging module, said at least one processor is configured to: receive said sensory data and said captured images; compare the captured images and the sensory data to a database to identify the characteristics of said substances.

According to a third embodiment there is provided a device for characterizing substances in a scene, the device comprising: a housing having a cavity therein wherein the cavity is configured to contain said substances; one or more openings for receiving said substances from the scene; an imaging module comprising one or more imagers or video imagers of high resolution for capturing one or more images of the substances; an illumination module comprising one or more illumination sources for illuminating the substances; a sensor module comprising one or more sensors for obtaining sensory data of the substances; at least one processor in communication with said sensor module and imaging module, said at least one processor is configured to: receive said sensory data and said captured images; compare the captured images and the sensory data to a database to identify the characteristics of said substances.

According to some embodiments, the processor comprises instructions to receive a sequence of images captured at different times and identify the substances by tracking one or more changes in the substances.

According to some embodiments, the substances are identified based on the movement of one or more microorganisms of said substances over time.

According to some embodiments, the substances are identified based on the way the one or more microorganisms interconnect to one other over time.

According to some embodiments, the processor comprises instructions to receive high-resolution video images from the video imager and identify the substances based on the high-resolution video images.

According to some embodiments, the processor comprises instructions to analyze the captured images to identify the shape and characteristic of one or more cells of the substances and characterize the substances based on the identified shape and characteristic of one or more cells.

According to some embodiments, the identification comprises identifying one or more colonies of the substances.

According to some embodiments, the identification comprises identifying the shape and size of said colonies.

According to some embodiments, the identification comprises identifying the movement of the substances' cells.

According to some embodiments, identifying the movements of the cells comprises one or more of: identifying the direction of the movement; its characteristic shape; its distance at a given time.

According to some embodiments, the identification comprises identifying the substances based on potential enemies and flight of microorganisms from them.

According to some embodiments, the identification comprises identifying the substances based on one or more of: where one or more microorganisms of the substances may develop; future evolution of the microorganisms; by comparison of the substances' microorganism to other well-defined microorganisms.

According to some embodiments, the identification comprises identifying the substances based said substances cells thickness.

According to some embodiments, the identification comprises identifying the substances based on said substances' cells measured Gram-positive or Gram-negative result.

According to some embodiments, the identification comprises identifying the substances based on the measured distance between cells or microorganism of the substances.

According to some embodiments, the identification comprises identifying the substances based on one or more molecules found in the cells of the substances.

According to some embodiments, the molecules are DNA or RNA.

According to some embodiments, the processor comprises instructions to capture images of the substances with illumination and with no illumination and compare the images to one another.

According to some embodiments, the one or more materials are added to the substances and the processor comprises instructions to track changes in the captured images and/or sensory data as a result of said materials insertion.

According to some embodiments, the one or more materials are antibiotics and the processor is configured to track the number of dead microorganisms in said substances to identify the substances.

According to some embodiments, the device comprising a gas supply device for storing and inserting gas into the housing.

According to some embodiments, the gas is one or more of: oxygen and/or nitrogen.

According to some embodiments, the device comprising a mechanical module configured to accordingly insert or take out the substances into or from the device via one or more openings.

According to some embodiments, the mechanical module comprises a mechanical arm.

According to some embodiments, the mechanical module comprises a syringe.

According to some embodiments, the device comprising a thermometer for measuring the temperature inside the device.

According to some embodiments, the device comprising a pump for creating vacuum in the device.

According to some embodiments, the device comprising an air pump configured to suck air from the housing and take out the substances from the housing.

According to some embodiments, the device comprising a fan for cooling the housing.

According to some embodiments, the device comprising a fluid injector injector for inserting fluid into the housing.

According to some embodiments, the device comprising one or more opening for inserting working tools to sterilely move or take the substances or control other modules in the device.

According to some embodiments, the working tools are gloves.

According to a fourth embodiment there is provided a security system comprising: a plurality of sensing devices for sensing substances in a toilet, wherein each device comprising: a sensing module comprising one or more sensors for sensing one or more substances in a toilet bowel and generating data on each sensed substance; and communication circuity; a plurality of imaging modules, each imaging module comprising; one or more cameras for recording images of said toilet users; communication circuity; a server; a computer-readable medium associated with the server, the computer-readable medium having stored thereon: said data of said substances; said toilet users images; computer-executable instructions for: analyzing the data of each substance to identify the type of said substance; linking said identified substance with said images to detect a threat object and the toilet user which said threat object belongs therein.

According to some embodiments, substances data comprises one or more of an identification of the substance, a classification of the substance among a plurality of classifications, one or more components of the substance, or categories of the substance.

According to some embodiments, said sensing module is embedded in a toilet cleaning device.

According to some embodiments, said sensing module comprises a processing module for identifying said sensed substances and generating diagnostic data of said sensed substances.

According to some embodiments, each of the sensing devices comprises an attachment module for attaching said sensing module to the toilet bowel surface or toilet bowel rim.

In another aspect, there is provided a toilet cleaning device comprising: a treatment module, said treatment module comprising: at least one treatment container, said at least one treatment container is configured and enabled to collect and store fluids, wherein said fluids are toilet flushing tank fluids; a first spraying module configured and enabled to spray said fluids and cleaning materials via a plurality of nuzzles into a toilet bowl; a sensing module comprising one or more sensors, said one or more sensors are configured to: sense one or more substances in the toilet bowel; and generate data on each sensed substance; a bridge module configured and enabled to be hanged on said toilet bowl rim and hold said treatment module away and below the toilet bowl rim at the path of the toilet flushing water, said bridge module comprising: a second spraying module configured and enabled to spray cleaning materials via a plurality of nuzzles on a toilet seat or toilet space; an external module, said external module comprising: at least one external container, said at least one external container is configured to store cleaning materials for cleaning or sanitizing said toilet; one or more pumps for delivering said cleaning materials to said treatment module; power source; and one or more processing modules for controlling the device modules and analyzing said data to identify said substances.

According to some embodiments, said one or more substances are one or more of a chemical; biological; radiological; nuclear; explosive agent, drugs; toxic, diseases microbes such as diabetes, cancer, infectious microbes.

According to some embodiments, said sensors are one or more of: sensors for detecting explosive agents; sensors for detecting weapons or weapon substances; sensors for detecting radioactive materials or radioactive indication substances; sensors for detecting diseases agents; sensors for detecting drugs; sensors for detecting alcohol; sensors for detecting DNA macromolecules.

### BRIEF DESCRIPTION OF THE DRAWINGS

For a better understanding of embodiments of the invention and to show how the same may be carried into effect, reference will now be made, purely by way of example, to the accompanying drawings in which like numerals designate corresponding elements or sections throughout.

In the accompanying drawings:

FIG. 1A shows an isometric upper side view of the cleaning device, in accordance with configurations;

FIG. 1B shows an isometric side view of the cleaning device, in accordance with configurations;

FIGS. 1C and 1D show another isometric side and top view of a cleaning device, in accordance with configurations;

FIGS. 1E-1F show another isometric view of the cleaning device, in accordance with configurations;

FIG. 1G shows an isometric top side view of the cleaning device 100 containers and covers, in accordance with configurations;

FIG. 2A shows a top side isometric view of the device hanged on a toilet bowl rim, in accordance with configurations;

FIGS. 2B and 2C show a side and top views of the device hanged on a toilet bowl rim, in accordance with configurations;

FIGS. 3A-3B show isometric views of the treatment module, in accordance with configurations;

FIGS. 3C-3D show an isometric upper side cross section views of the treatment module, in accordance with configurations;

FIGS. 3E-3F show an isometric outward top and side views of the treatment module, in accordance with configurations;

FIG. 3G shows an isometric view of the treatment module inner modules, in accordance with configurations;

FIG. 3H shows a view of the inner modules of the treatment module, in accordance with configurations;

FIG. 3I shows a rotatable external arm, in accordance with configurations;

FIGS. 4A-4D show a number of isometric views of the storage and control module, in accordance with configurations;

FIGS. 5A and 5B show an upper side isometric views of the bridge module, in accordance with configurations;

FIG. 5C shows an upper isometric view of the rotating wheels inside the treatment module, in accordance with configurations;

FIG. 5D shows an upper isometric view of the rotating wheel, in accordance with configurations;

FIG. 6 is a block diagram illustrating a block diagram side view of a device for detecting and identifying substances such as explosive or toxic substances in toilets, in accordance with embodiments;

FIGS. 7A-7B illustrate a block diagram of a system for detecting, diagnosing and alerting on chemical-biological-explosive (CBE) or other threats on-site and allow for immediate responders to mitigate spread, risk, and loss, in accordance with embodiments;

FIG. 7C illustrates a device positioned in proximity to the toilet compartments, in accordance with embodiments;

FIG. 8 is a flowchart of a method for detecting diagnosing and alerting on substances threats on-site and allow for immediate responders to mitigate spread, risk, and loss, in accordance with embodiments;

FIGS. 9A-9B illustrate an isometric top view of a cleaning and detection device, in accordance with embodiments;

FIGS. 10A-10B illustrates a diagram of a system for detecting, diagnosing and alerting on chemical-biological-explosive which is hanged on a toilet bowl rim, in accordance with embodiments;

FIGS. 10C-10D illustrate a diagnosing module included in the external module housing, in accordance with embodiments;

FIG. 11 is a flowchart illustrating a method for identifying and/or diagnosing one or more substances in a toilet in accordance with embodiments;

FIG. 12 is a flowchart of a method for diagnosing and/or recognizing one or more substances found within a toilet bowl, in accordance with embodiments;

FIG. 13A-13E show isometric views of different devices for characterizing and/or diagnosing one or more substances in a scene, in accordance with embodiments;

FIG. 14A shows a flowchart of a method for characterizing and/or diagnosing one or more substances or objects in a scene using one or more sensors and imagers having high resolution capabilities, in accordance with embodiments;

FIGS. 14B-14U illustrate one or more visual examples of the diagnostic methods used while analysing the captured images and/or video images of the substances as mentioned herein in respect to flow chart steps illustrated in FIG. 14A, in accordance with embodiments; and

FIG. 15 illustrates one or more examples of insertion modules which may be attached or included in the device, in accordance with embodiments.

### DETAILED DESCRIPTION OF THE INVENTION

As explained above, the present invention relates generally to the detection and diagnosing of substances such as infectors, drugs or toxic compounds and more particularly, to a devices systems and methods for detecting and alerting of threats found in toilet or toilets compartments such as infectors explosives drugs or toxic compounds.

As used herein like characters identify like elements.

As used herein the term ‘toilet’ encompasses a device that may be used to collect one or more biological waste products of a user.

As used herein the term ‘user’ encompasses human or animal that deposits bodily waste into an embodiment of the toilet disclosed herein.

As used herein the term ‘Object Under Test’ (OUT) encompasses one or more substances and/or objects such as a biological/chimerical substance in a scene.

The examples disclosed herein can be combined in one or more of many ways to provide improved substances or an OUT such as explosive and/or drug detection, identification and alert systems, devices and methods positioned for example in toilets for example on a toilet bowl or seat.

A variety of detection systems and devices such as closed-circuit cameras and sensors were developed to monitor in real-time attempts and provide intelligent on terror suspects. While the potential saboteur and terror organizations are well aware and cautious of security devices and means such as cameras, sensors and police troops in public surroundings they are less careful and their behavior is sometime reckless in private areas such as public toilets compartments.

There are therefore provided in accordance with embodiments a detection and alert devices, systems and methods comprising an attachment module such as a hanging module configured to be suspended or attached to a toilet bowel or a toilet seat. The hanging module holds one or more modules comprising one or more sensors for sensing substances such as chemical and/or biological and/or explosive substances or compounds and provide sensory data of the substances and one or more imagers for capturing high resolution images or video images of the substances. The systems and devices may further include or may be in communication with one or more processors configured to receive data including the captured images and the sensory data from the one or more sensors and imagers and analyze the data to identify the type of sensed substances and transmit information on the identified substance via one or more communication modules.

In accordance with another embodiment, there are provided devices, systems and methods for capturing images of one or more substances in a scene using an illumination module and an imaging module comprising one or more high quality imagers configured to capture one or more images or an image video of the substances and a processor for analyzing the captured images to identify the characteristic and/or type of the substances. In some cases, the devices may be portable. For example, the imaging module may be included (e.g. attached) in a portable housing having a cavity configured to contain the one or more substances. In some cases, the imagers may be or may include optical devices configured to image microscopic substances or elements. In some cases, the substances are diagnosed by comparing the captured images to images or data stored for example at a local or foreign database. In some cases, the images are analyzed using one or more of: neural network (e.g. Deep Neural Network (DNN)); Artificial Intelligence (AI). In some cases, the substances are identified by monitoring the substances over time and analyzing the movement, behavior (e.g. what they eat), shape (e.g. shape and/or type and/or structure of a signal cell or cell colony) of the substances. In some cases, the substances may be or may include microorganisms such as bacterium, virus, or fungus; Nano particles or subatomic particles of various types such as materiel used explosive or toxic substances, infectors, drugs, microbes such as infectious microbes and the like, in accordance with embodiments.

In an embodiment, the captured images of the substances can be used to determine one or more attributes of the substances. In many embodiments, the devices and systems, such as the housing is coupled to a database including information that can be used to determine the attributes of the substances. The systems and devices may comprise a hand held communication device coupled to the housing, in which the user can input and receive data related to the measured substances with the hand held communication device.

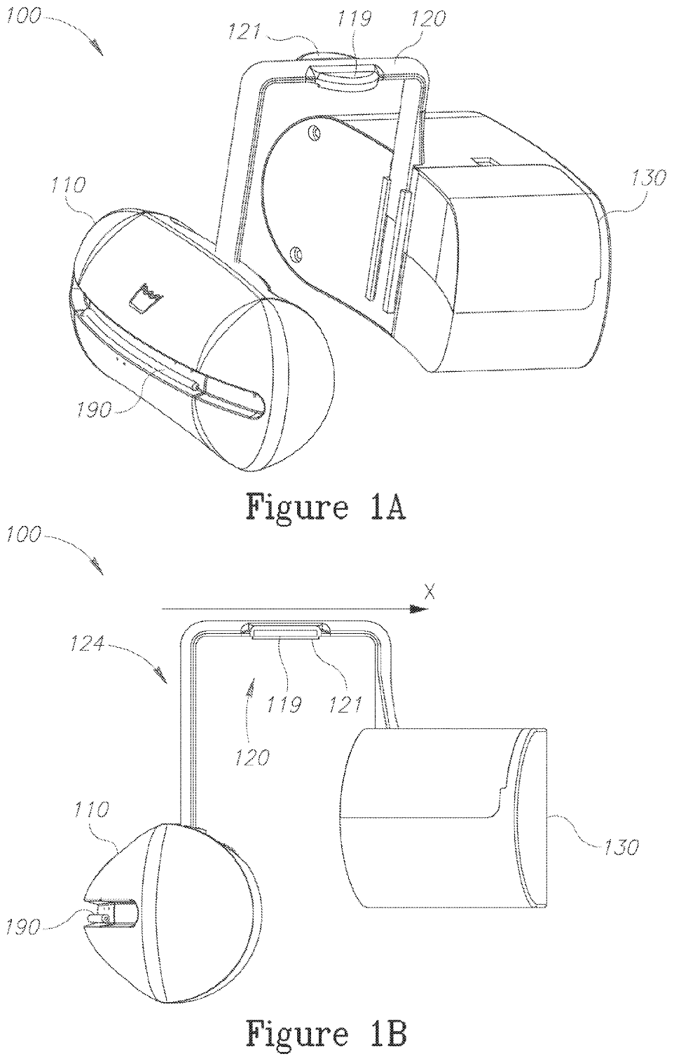

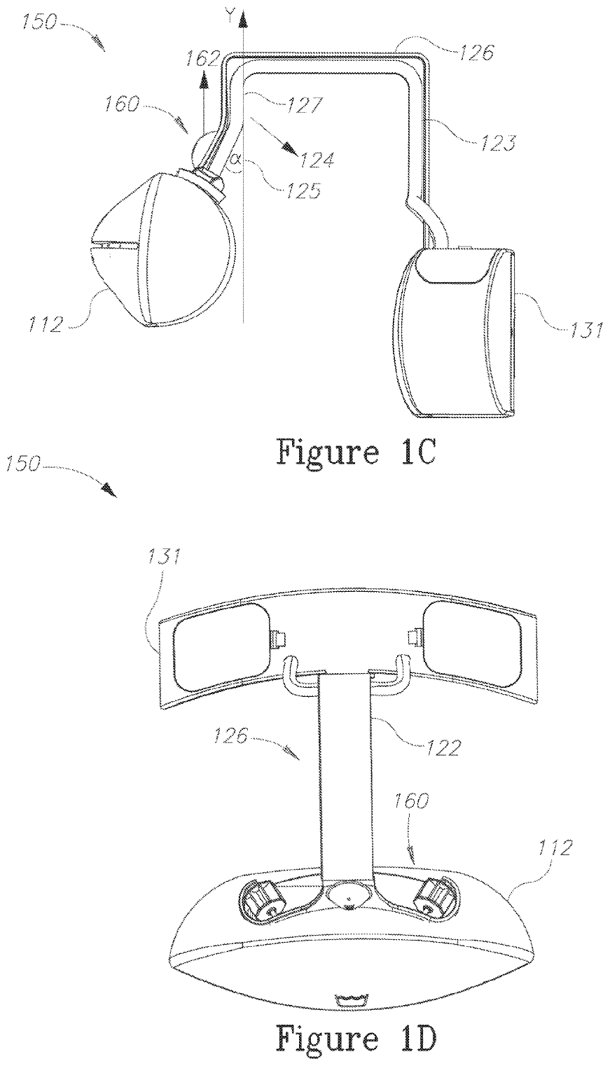

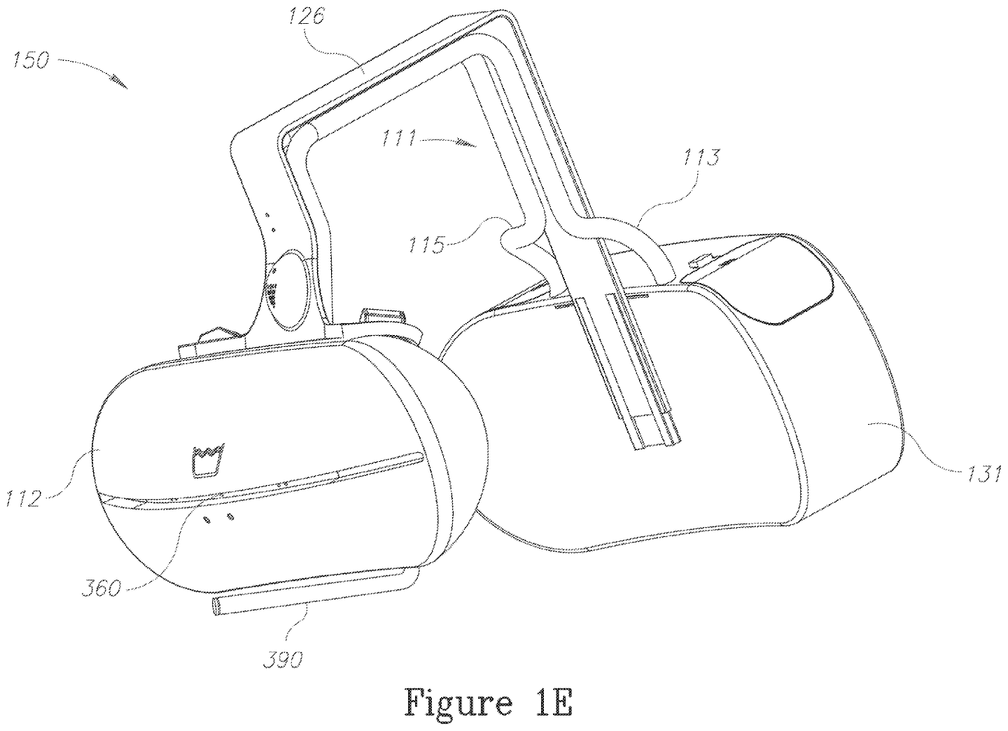

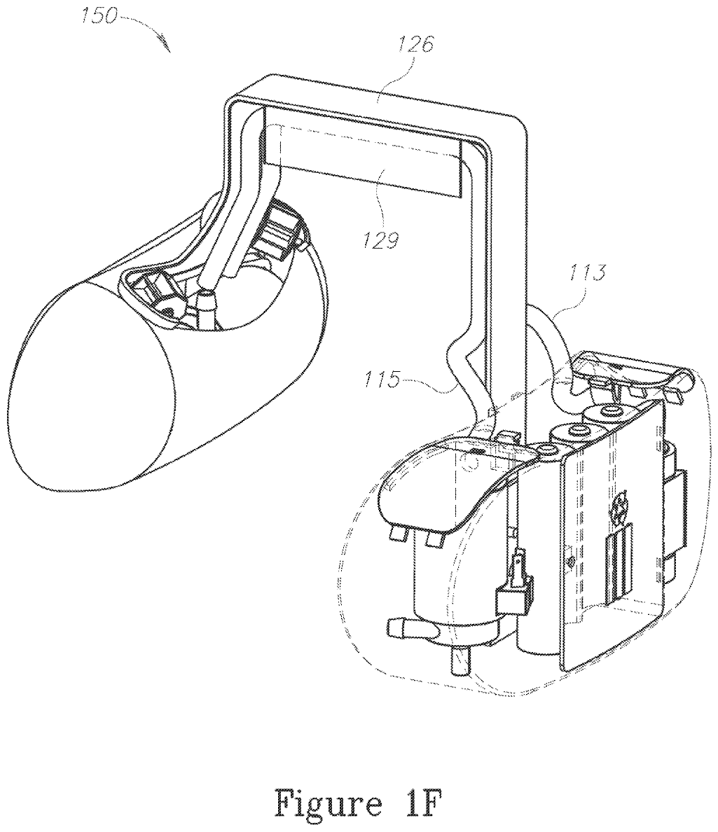

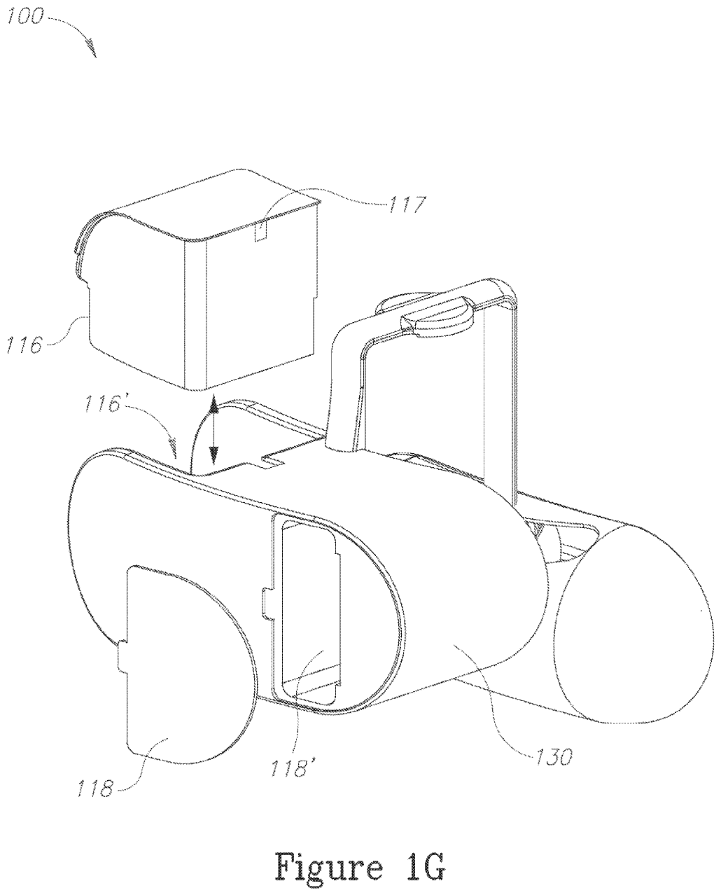

Reference is made to FIGS. 1A-1B illustrating a cleaning device 100 for automatically and/or autonomously cleaning toilets, in accordance with some embodiments. FIG. 1A shows an isometric upper side view of the device 100, FIG. 1B shows an isometric side view of the device 100. According to some embodiments, the device 100 may be modular including for example three main modules: a treatment module 110, a bridge module 120 and a storage and control module 130.

In some cases, the three modules are formed and connected as of one piece, alternatively the treatment module 110 and the bridge module 120 are formed or connected together as one piece and the bridge module is connectable to the storage and control module 130.

In accordance with embodiments, the treatment module 110 may comprise one or more fluid reservoirs or containers, cleaning agents and materials, one or more cleaning and spraying modules. The treatment module 110 is configured to collect and receive fluids such water flushing from the toilet's flush tank and cleaning treatments such as detergents received for example from the 130 or cleaning materials placed at the treatment module 110, and form a chemical mixture from the received fluids and cleaning treatment. The chemical mixture is then sprayed for example automatically along the perimeter surface of the toilet bowl.

According to some embodiments, the treatment module 110 comprises a spraying arm 190, shaped for example as an elongated pipe. In some cases, the spraying arm 190 is positioned externally to the treatment module and is configured to rotate in perpendicular to the treatment module 110 length. In some cases, the spraying arm 190 is configured to deliver and disperse cleaning material, such as materials including sanitization agents from the storage and control module 130 and spray the materials into the interior of the toilet.

In accordance with embodiments, the device 100 comprises hanger means such as a bridge module 120 (e.g. suspension module) configured to be hanged for example on a toilet bowl rim and hold the treatment module 110 away from the toilet bowl, for example below the toilet rim at the path of the flushing water, thus water flushing during flushing operation on the sidewalls of the toilet bowl surface will be efficiently accumulated by the one or more fluid reservoirs or containers of the treatment module 110.

The cleaning device 100 further comprises a storage and control module 130 for storing cleaning materials or compositions such deodorizing and disinfecting materials (e.g. detergents) which may be dispensed by the treatment module 110 into the toilet bowel.

In some cases, the storage and control module 130 includes control and processing means such as one or more processing modules for processing data received from one or more sensors of the device and/or external sensors which are in communication with the device 100 and/or external devices such as computer devices or smart mobile phone devices or the like. Based on the processed data the processing modules are configured to operate and control the device modules, such as the spraying modules.

In some cases, the device, for example the storage and control module 130, may include one or more timers for activating the device modules, such as the one or more spraying modules.

According to some embodiments, the sensing modules may include one or more sensors such as proximity sensors and/or pressure sensors for detecting a movement of a user at the device's vicinity and accordingly activating or deactivating the device. The sensors may be for example RF sensors, IR sensors, pressure sensors, laser sensors. In some cases, the sensors may be sensors configured and enabled to detect the smell in proximity to the cleaning device. In some cases, the sensors may be sensors of Interlink Electronics FSR™ 400 series. The sensors may include Force Sensing Resistors, or FSRs. The sensor may be a round sensor of 18.28 mm in diameter.

In some cases, the proximity or motion sensor may be HC-SR505 Mini PIR motion sensor or other known motion sensors which are based on infrared technology and may automatically control by itself with high sensitivity and high reliability. (see for example http://www.elecrow.com/hcsr505-mini-pir-motion-sensor-p-1382.html)

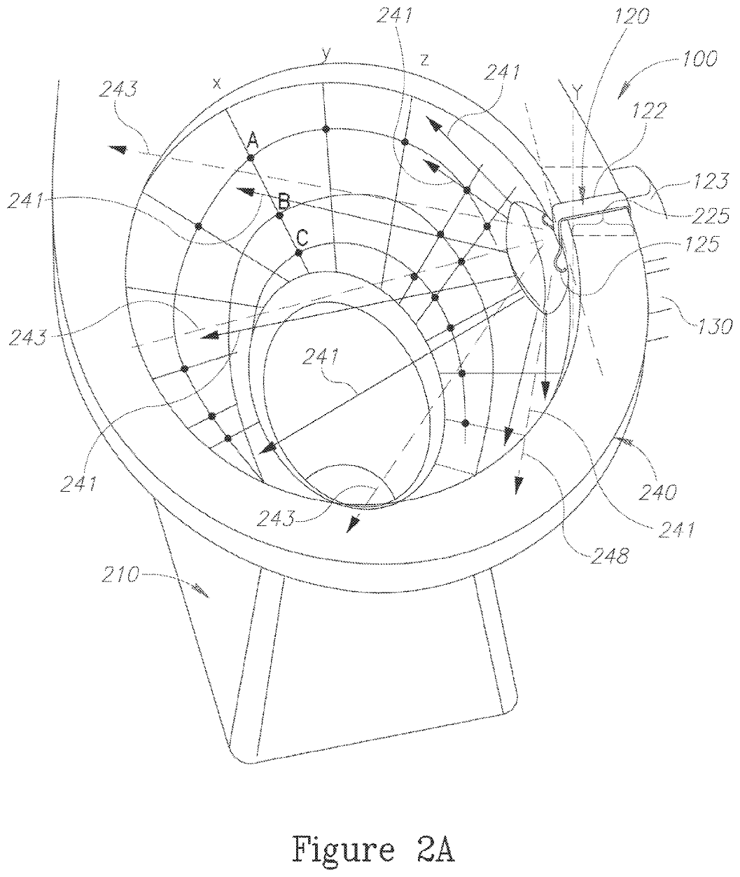

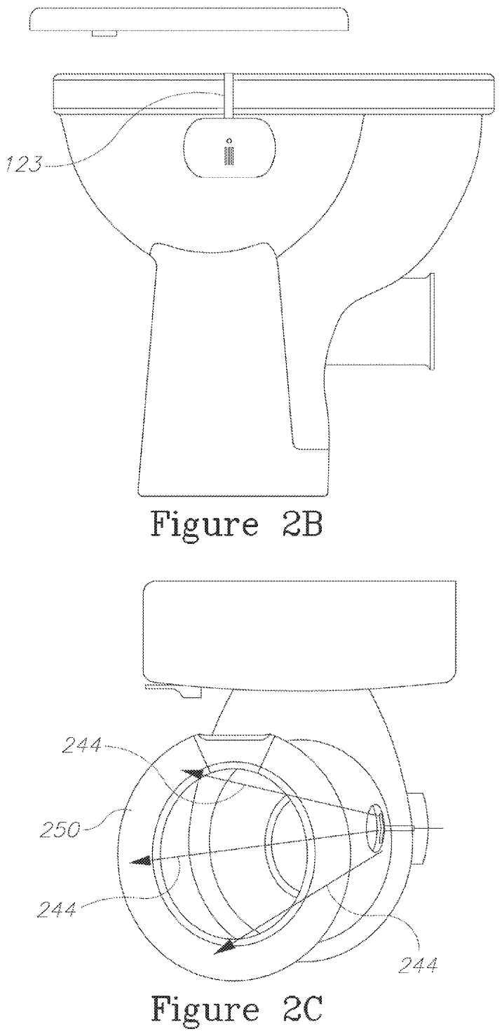

In some cases, the storage and control module 130 are positioned exterior to the toilet bowel, for example below the toilet bowl rim as shown in FIG. 2A-2B. In some cases, the external module may be placed in any other location.

In accordance with embodiments, the bridge 120 is made of strong and flexible materials such as plastic, polymer, still or any known elastic material.

In accordance with other embodiments, the bridge 120 maybe made of one piece and the bridge portions extending from the sides of the bridge base may be perpendicular or substantially perpendicular (e.g. around 90-60 degrees) in respect to an X axis.

In accordance with some embodiments, the bridge may include an elongated section, for example extending from the center section 121 of the bridge having a rounded shape for stabling and holding the device on the toilet bowel rim.

In accordance with some embodiments, one or more sensors may be embedded in the bridge module such as a proximity sensor, pressure sensor or a sensor configured to identify or be activated once contact or pressure is identified. For example, a sensor 119 may be embedded at the center section for identifying and sensing pressure or movement of a user such as a user seating on the toilet seat.

In accordance with another embodiment, one or more sensors such as a proximity sensor 114 may be attached to the treatment module 110 for example at the top side of the treatment module 110 or may position within the treatment module for identifying movements in proximity to the device.

Reference is made to FIGS. 1C-1D illustrating a cleaning device 150 for automatically and/or autonomously cleaning toilets, in accordance with another embodiment. FIG. 1C shows an isometric side view of the device 150, FIG. 1D shows an isometric top view of the device 150.

According to some embodiments, the device 150 includes a power generator module 160 for converting mechanical energy into electrical current. In some cases, the power generator module 160 may be connected for example to bridge module 126 and is further configured to transfer and/or form power for recharging the device's power source (e.g. batteries) as will illustrated herein below.

According to some embodiments, the bridge 126 comprises one or more indication modules 162 such as one or more warning and status lights or small speakers to indicate the device 150 modes, e.g. sleep/active mode etc.

FIGS. 1E-1F show other isometric views of the cleaning device 150, in accordance with embodiments. The cleaning device 150 comprises one or more pipes 111 for delivering the cleaning compositions from the storage module 131 to the treatment module 112 for example for supplying cleaning agents and/or fluids to the spraying modules such as sparing module 360 and/or spraying arm 390. In some cases, the pipes 111 may be external or partially external to the device 150. According to some embodiments, the cleaning device 150 may comprise two separated pipes. A first pipe 113 may be connected at one end to one of the device's containers for example to a first container such as container 479 comprising liquid and a second pipe 115 may connect at one end to a second container comprising foaming material. The other ends of the first and second pipes may be attached or coupled to the inner portion of the treatment module 112.

The external portions of the pipes 113 and 115 may be coupled or attached to the back side of the storage module 131 and to the inner section of the bridge 126. According to some embodiments, the pipes are flexible and the external portions of the pipes are bended along the bridge shape to avoid overhanging. The pipes are further inserted to the treatment module and are connected vie one or more openings to the pumps for spraying the fluids and/or cleaning materials to the toilets or to the toilets surroundings.

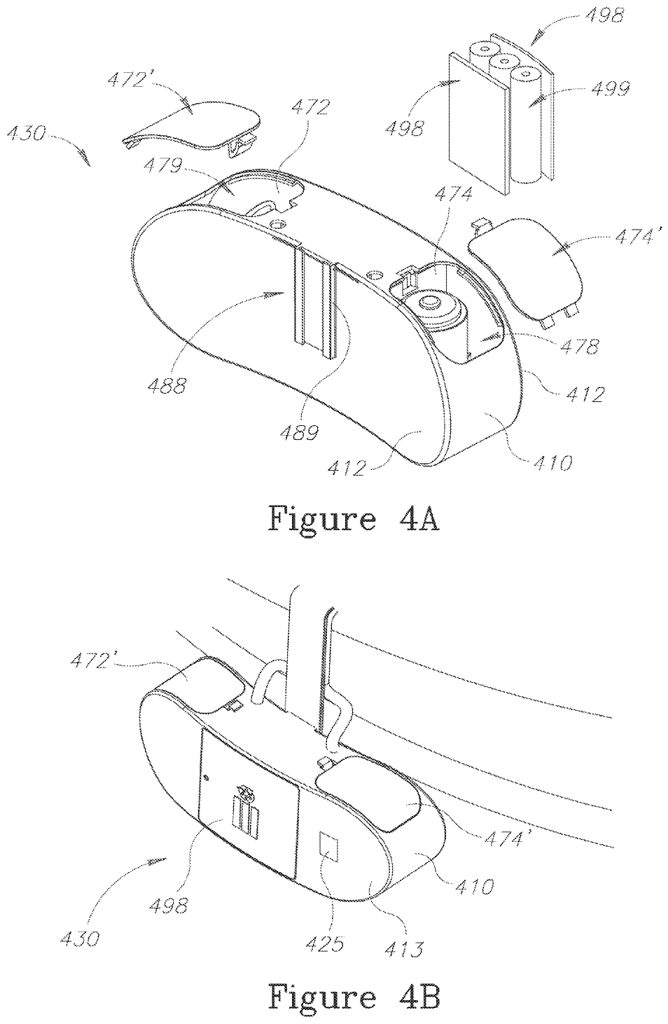

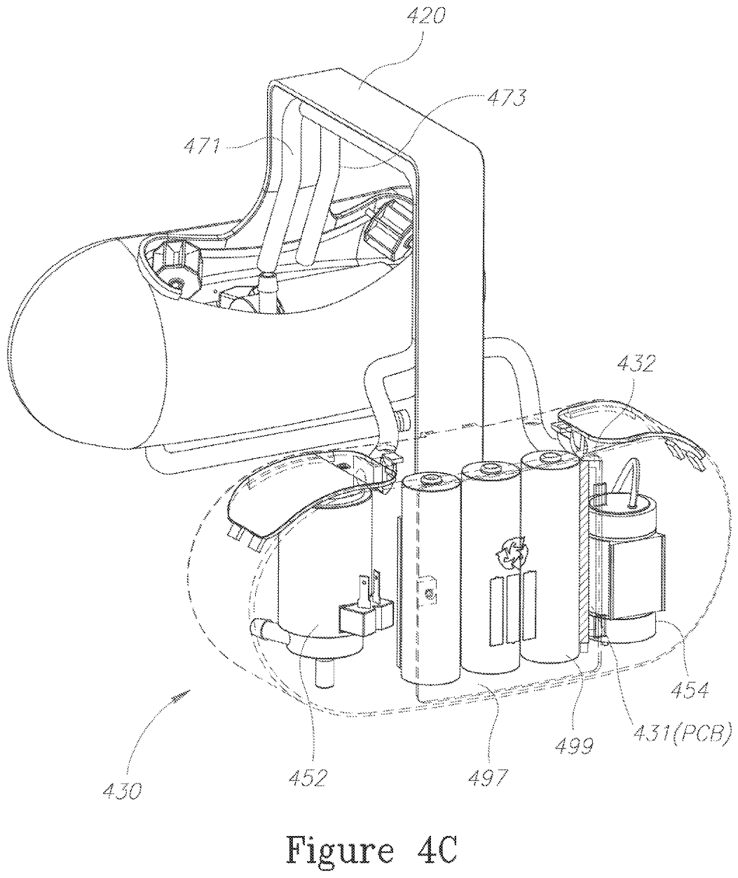

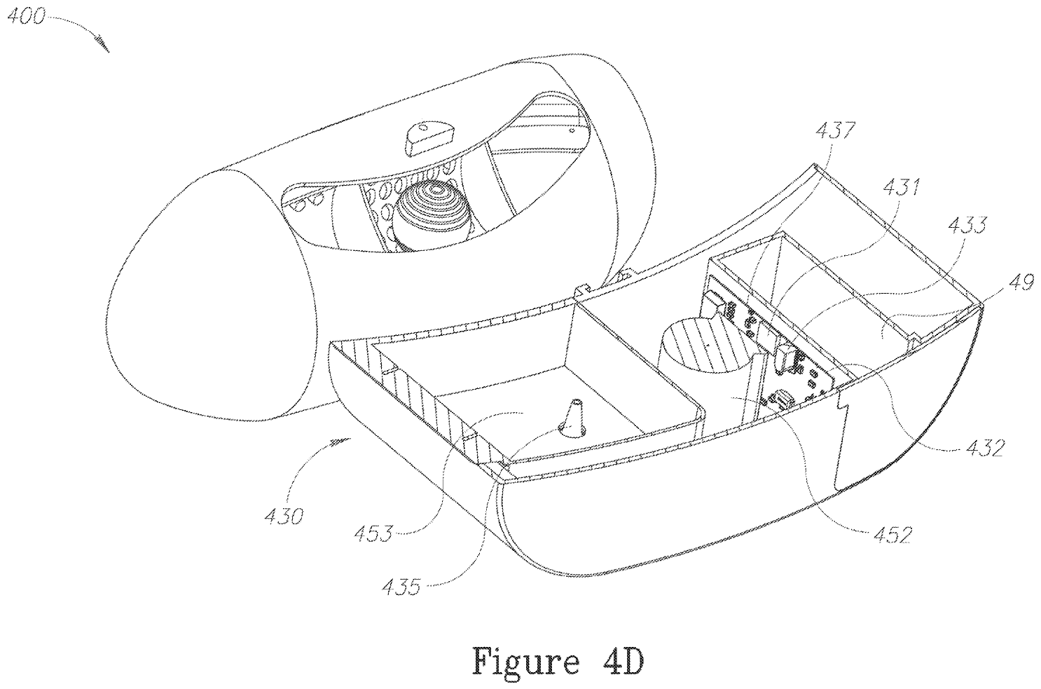

According to some embodiments, one of the pipes such as pipe 113 may deliverer sanitization materials to the external sparing arm 390 via one or more pipes for sparing the interior of a toilet as illustrated in FIG. 4K, while the other pipe such as pipe 115 may deliver cleaning material which may be mixed with fluids aggregated at the treatment module and sprayed via one or more pipes and nozzles of spraying module 360 for cleaning the toilet bowl surface as illustrated in FIG. 2A.

According to some embodiments, as shown in FIG. 1F the bridge 126 may comprise a hanging protection 129 located under the flat stripe section to avoid the pipes crushing by the toilet seat or to avoid any unwanted pressure on the pipes.

In some cases, the bridge 126 may comprise a double sealing cover wall and the pipes may be placed inside the cover. In some case, a ‘built in’ bridge comprising one or more pipes may be provided.

In some cases, the pipes may be made of plastic or other materials as known in the art.

FIG. 1G shows an isometric top side view of the cleaning device 100 containers and covers, in accordance with embodiments. In some cases, the device modules such as the batteries and cleaning containers may be easily releasable from the device and inserted back to the device. For example, the device may include a container such as an aerosol container 116 which may include a frame and audible or physical “click” type confirmation portion 117 or other modules which can positively engage the container 116 to the matching cavity 116′ to ensure that a suitable seal has been secured.

In some cases, the storage and control module 130 may include a releasable cover 118 for covering for example from the side one or more batteries or other elements stored in the storage and control module 130 such as at cavity 118′. In some case, the cover 118 and the container 116 may include a printed sign on the image explaining the type element placed within the cover of container, e.g. batteries, aerosol, etc.

FIG. 2A shows a top side isometric view of the devices 100 or 150 of FIG. 1 hanged on a toilet bowl rim 240 in accordance with embodiments. In some cases, the devices 100 or 150 may be suspended from the toilet rim 240 by hanging the bridge module 120 for example along the surface of section 225 of the toilet bowl rim 240.

According to some embodiments the device modules, such as the bridge 120 are adjustable and configured to be placed or attached to any type or size of toilets.

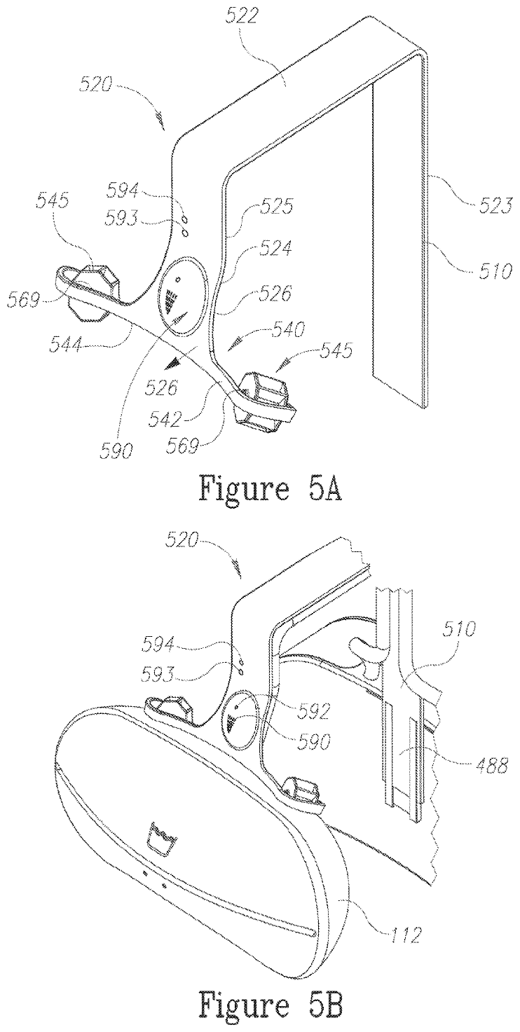

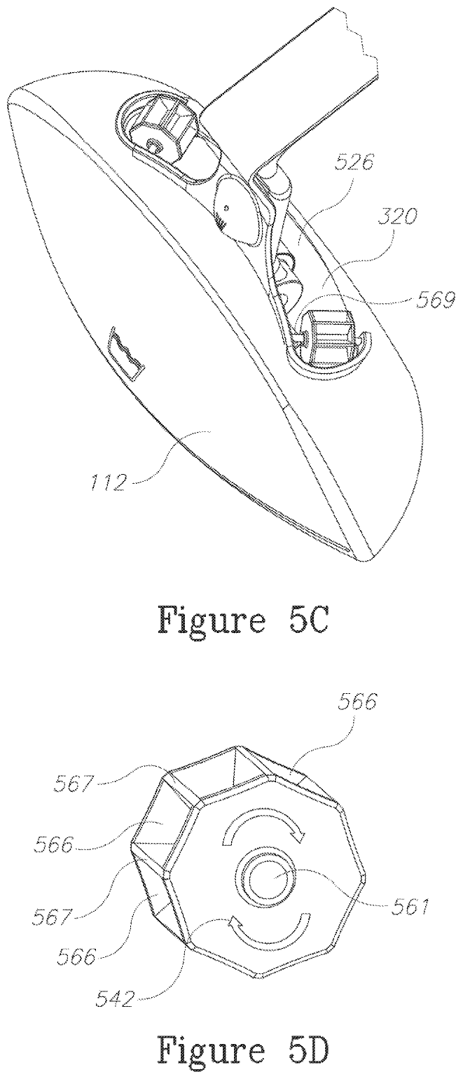

Specifically, the bridge module 120 comprises a flat stripe section 122, preferably to accommodate the geometries of different types of toilet bowls and two flexible sections, extending from the flat stripe section, configured to be folded along the inner and external sides of the toilet bowl rim 240. For example, as shown in FIG. 1C, a first flexible section 123 extending from section 122 may be bended in perpendicular or substantially in perpendicular (e.g. between 60-90 degrees) in respect to the toilet bowl rim surface 240 downwardly towards the outer side of the toilet bowl rim surface. The first flexible section 123 may be connected to the storage and control module 130 and hold the storage module 130 in parallel or below the toilet bowl rim surface 240 as illustrated in FIGS. 2B and 2C. The bridge module 120 further comprises a second strip section 124 extending from the other end of the flat stripe section 122 downwardly towards the interior of the toilet bowl rim. The second flexible section 124 may be connected and hold the treatment module 110 preferably below and away from the toilet bowl rim 240. As shown in FIG. 2A and further illustrated in FIGS. 5A and 5B a first portion 127 of the second strip section 124 extending from the flat stripe section 122 is bended vertically in respect to the flat section 122 while a second portion 125 extending from the first portion 127 is bended outwards in angle a of between 0-45 degrees in respect to axis Y from the toilet bowl rim to place the cleaning module below and away from the rim 240, for example at the path of the flushing water. Thus water flushing on the sidewalls of the toilet bowl inner surface during flushing operation will be efficiently accumulated or to at least temporarily will retain a number of flushing fluids at the treatment module 110.

In some cases, as illustrated in FIG. 2C, once the device is positioned on the toilet bowel rim, the device may be covered by the toilet's seat 250 to avoid direct contact between the device 100 and a user.

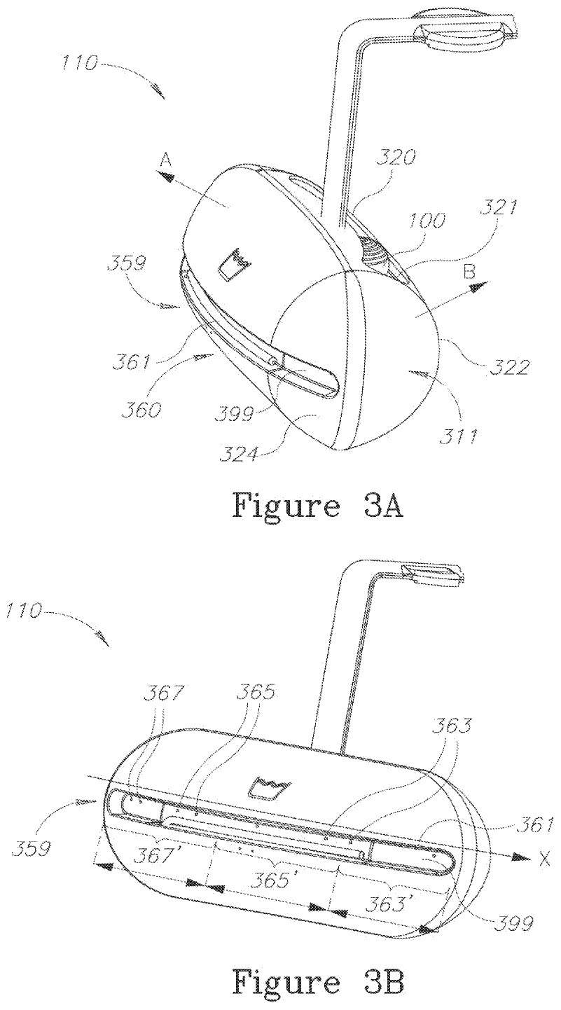

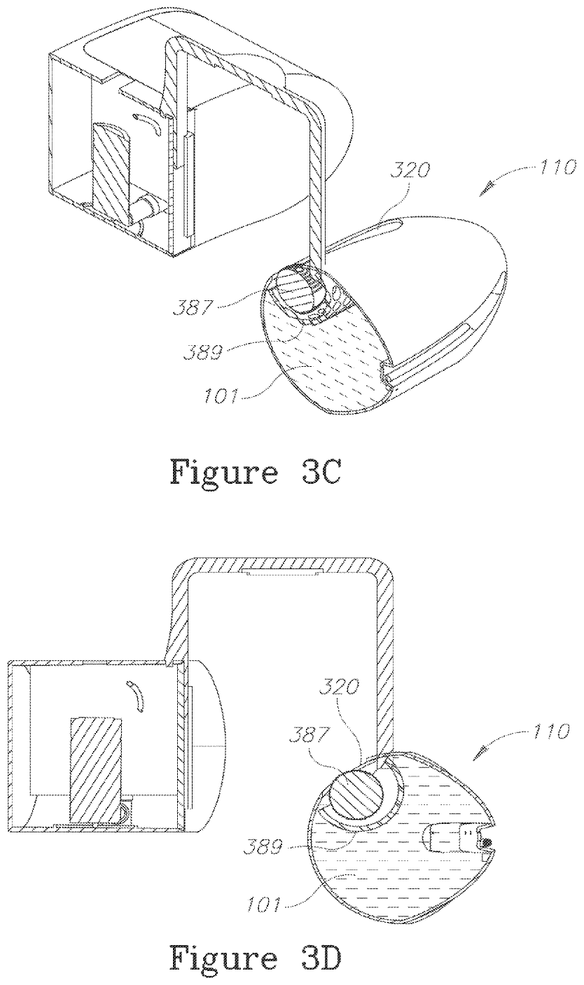

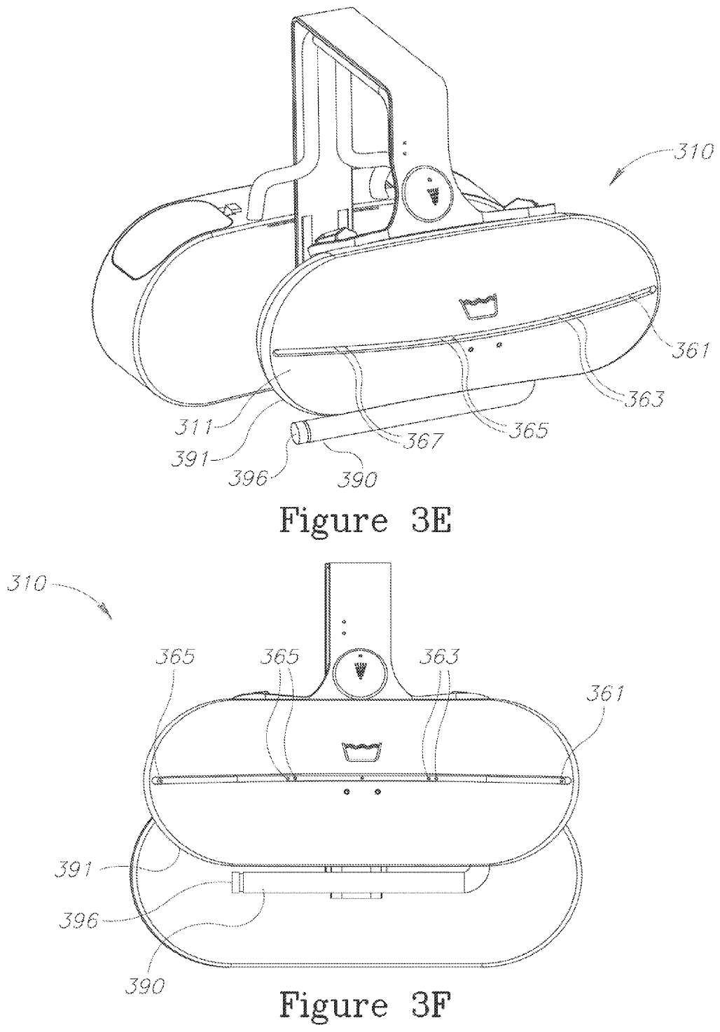

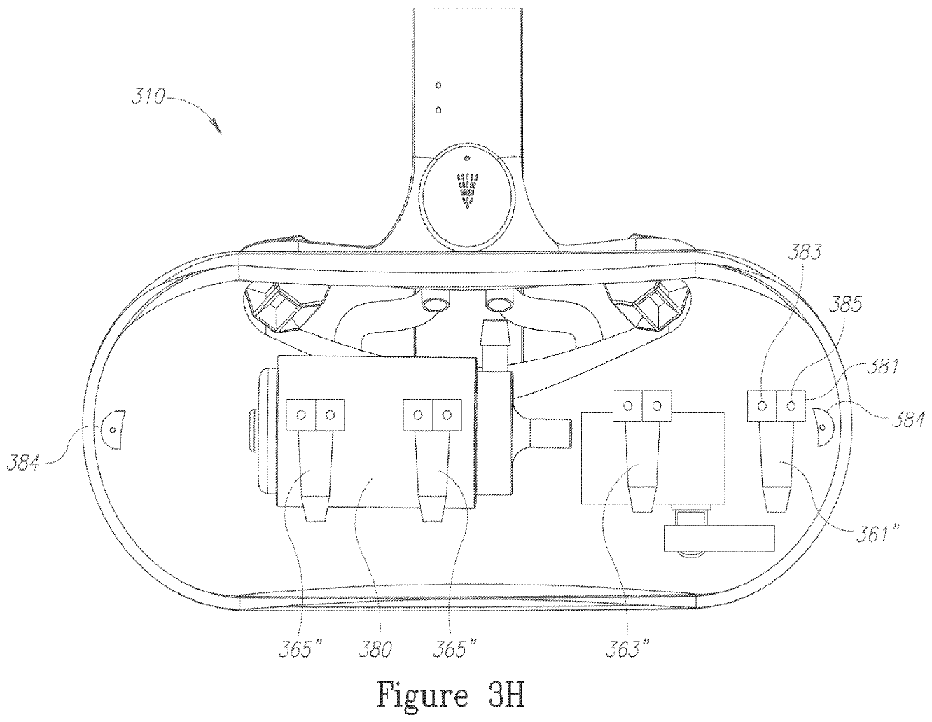

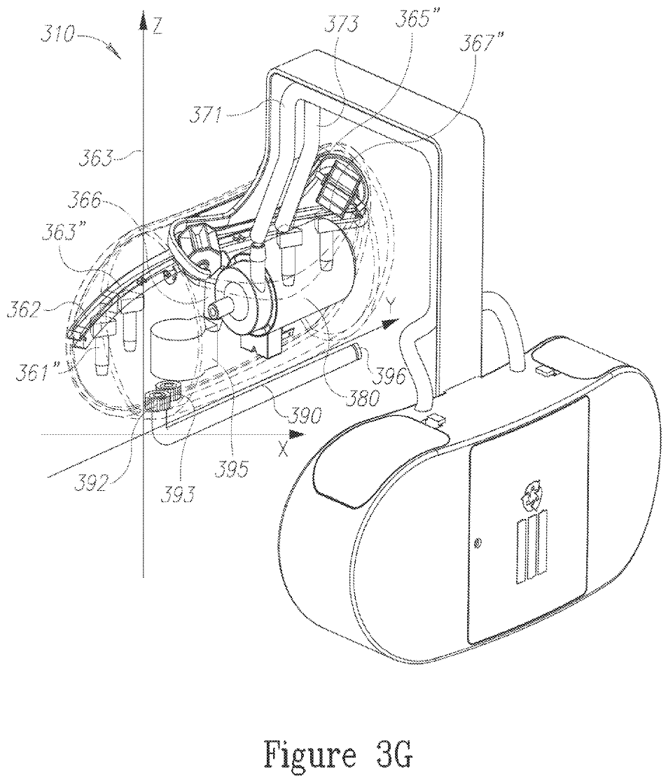

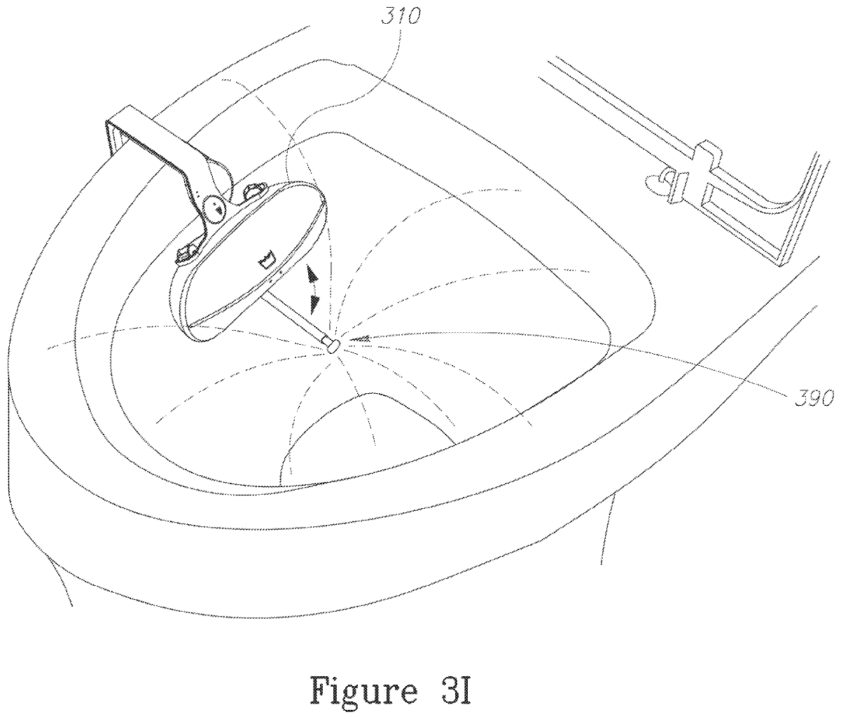

Reference is now made to FIGS. 3A-3J illustrating a number of isometric views of the treatment module 110, in accordance with a number of different embodiments.

As illustrated in FIG. 3A, the treatment module 110 comprises a housing 311 for covering and holding one or more fluid reservoirs such as container 321 placed within the housing. The container 321 is configured to accumulate fluids such as fresh water flushing from the toilet's flush tank via one or more openings or apertures, for example through opening 320.

According to some embodiments the opening 320 may be formed at the top right side of the housing and may have a crescent shape. In some cases, the opening width size may be between 10-30 mm for example 18 mm. In some cases, the opening may have other shapes and size.

The housing 311 may include or may be made of a number of cover parts such as two cover parts 322 and 324 which may be attached to one another according to methods as known in the art (e.g. vacuum pressure, glue, or attachment pins 384, etc.). According to some embodiments, the housing 311 is convexly shaped to enable fluid such as fresh water flushing from the toilet's flush tank to gather and flow into the opening 320 or flush down the toilets.

According to some embodiments, the housing may be made of one piece having for example two convexly shaped sides A and B.

The treatment module 110 further comprises one or more spraying modules such as a first spraying module 360 (e.g. bottom spraying module) for dispersing cleaning composition and/or fluids such as flushing tank fluids (e.g. fluids accumulated at the container 321). The first spraying module 360 is configured and enabled to spray the fluids typically with a cleaning composition (e.g. the detergent) all over the interior toilet bowl perimeter according to a number of predetermined intervals till a complete cleaning and sanitization of the toilet bowl is accomplished.

In this embodiment, an external arm 361 is located an elongated groove 399 forming a long narrow channel along with the treatment module 110 length, for example at the center or substantially the of the treatment module. In some cases, the external pipe is folded and extends out of the housing 311 via opening 361.

In operation, the external pipe rotates out and away from the treatment module, in perpendicular (e.g. between 0-120 degrees) to the treatment module length (e.g. X axis) for spreading sanitization material in 360 degrees (all directions) at the entire cavity or substantially the entire inner cavity and outer surroundings of the toilet bowel as illustrated in FIG. 3F.

According to some embodiments, the spraying module 360 is located at an elongated groove 399 formed (e.g. notched) at the center or near the center of the treatment module 130.

In some cases, the elongated groove 399 may be formed longitudinally at the outer surface of the housing at side A on the front side of the treatment module facing the toilet bowl inner surface at the center of the housing around a perimeter, for example at the center, or substantially near the center of the treatment module 110.

In some cases, the groove 399 may be shaped like a rectangle along and within the housing having a depth of 1-40 mm.

For example, as illustrated in FIG. 2A a number of streams (i.e. arrows 241) are directed by the spraying module 360 all over the interior toilet bowl perimeter to efficiently clean the toilet bowl. According to some embodiments, the elongated groove 399 includes a rail 362 notched along the housing 311 (shown also in FIG. 3G) and one or more nozzles are embedded or coupled within the rail 362 inside the housing 311. In some cases, one or more nozzles are configured to move back and forth along the rail 362 and/or rotate while spraying cleaning composition to the toilets. In some cases, the rail 362 may be divided to a number of segments and the nozzles may be embedded along with these segments. For example, as illustrated in FIG. 3B the spraying module 360 may include a number of openings such as four openings 361, 363, 365 and 367 formed along the groove 399 for enabling a number of respective nozzles 361″, 363″ 365″ and 367″ located inside the treatment module housing to spray cleaning materials (e.g. detergent materials) to the interior of a toilet. In some cases, the nozzles are fixed, for example to the rail 362. In some cases the rail 362 may respectively be divided to three or more sections such as sections 363′, 365′ and 367′ and the nozzles may move back and forth along the segments spraying for example concurrently a number of sections of the toilet bowl.

In some cases, each of the nozzles 361″ 363″, 365″ and 367″ are configured for facilitating dispersion of fluids aggregated in the housing 311 into a spray. The nozzles are used to distribute the aggregated fluids mixed for example with cleaning materials over the toilet bowl area, to increase liquid surface area, and create impact force on a solid surface.

In some cases, the nozzles may be spray nozzles such as solid-cone single-fluid nozzle, compound nozzle and other nozzles as known in the art.

According to some embodiments, the nozzles may move and rotate along the rail by one or more motors such as one or more step motors or by fluid pressure means.

In some cases, the nozzles may move and rotate autonomously without the use of a dedicated motor.

In some cases, the nozzles may move or rotate by one or more means configured to move and rotate the nozzles such as one or more springs, fluid pressure means, chemical reaction, air pressure, etc.

In some cases, the treatment module 110 may include a single nozzle which is configured to move along the rail back and forth.

In some cases, the spraying module may spray the accumulated fluids with or without the cleaning composition (e.g. the detergent) all over the interior toilet bowl

According to some embodiments, the treatment module 110 includes an additional spraying module 359 configured to spray cleaning materials such as sanitization and/or foaming material into the interior of a toilet bowl, for example simultaneously in all directions (e.g. 360 degrees) as shown in FIG. 3I. The spraying module 359 may include, for example, an elongated arm in the form of an elongated cylindrical pipe which may be placed for example in the elongated groove 399. In a pause mode, the pipe is hidden within the groove. Once activated the pipe is rotated outward, for example in perpendicular to the treatment module length axis.

According to some embodiments, the treatment module 110 length may be between 100 mm to 200 mm for example 170 mm long and height of between 50 mm to 100 mm, for example, 71 mm. The cylindrical pipe 361 may be between 50 mm to 100 mm for example 90 mm long and the groove 399 may be for example between 60 mm 190 mm long for example 160 mm long.

Reference is now made to FIGS. 3C-3D illustrating an isometric upper side view of the treatment module 110. FIG. 3C shows an isometric cross section of the treatment module, while FIG. 3D shows an isometric external side view of the treatment module 110. In accordance with embodiments, the treatment module 110 includes one or baskets such as basket 389 for holding a solid cleaning material 387. In some cases, the basket 389 may include a plurality of holes for enabling fluids 101 flowing into the treatment module cavity via opening 320 to be mixed and dissolve with the cleaning material 387. Specifically, in operation, the fluids 101 stored at the housing cavity are mixed with the solid cleaning material 387 forming cleaning fluids which are later sprayed for cleaning the toilets (e.g. toilet bowel or toilet surrounding).

FIGS. 3E-3H illustrate a treatment module 310, in accordance with another embodiment. Specifically, FIGS. 3E-3F illustrate respectively an isometric outward top and side views of the treatment module 310 while FIG. 3G shows an isometric view of the treatment module 310 inner modules, and FIG. 3H shows a view of the inner modules of the treatment module 310, in accordance with other embodiments. The treatment module 310 includes one or more pumps such as pump 380, inlet and outlet pipes such as pipes 371 and 373, one or more nozzles such as spraying nozzles 361″, 363″, 365″, 367″, one or more motors and engines such as motor 395, cogwheels 392 and 393, and an exterior spraying arm 390 and one or more sockets for holding the treatment module 310 inner modules.

According to some embodiments, the one or more pumps such as pump 380 are configured and enabled to pump (e.g. suck) fluid accumulated and stored at the housing 311 cavity via one or more inlet pipes into the pump 380 and from the pump 380 to the nozzles 361″, 363″, 365″, 367″ for spraying the pumped fluids to the toilet bowl (e.g. into the interior of a toilet) via respective openings 361, 363, 365 and 367. In some cases, the fluids include flushing tank fluids, such as fluids aggregated at the container 321 or at the treatment module 310 cavity mixed with cleaning materials transferred via pipe 371 from the storage and control module 130 or with cleaning materials such as solid or fluids cleaning materials placed in the treatment module cavity.

According to some embodiments, one or more external pipes such as the external arm 390 may be attached to the bottom outer side of the treatment module 310, for example to the outer lower surface 391 of the treatment module 310 and connected via one or more pipes such as pipe 373 for delivering the one or more materials or compositions, such aerosol and/or disinfection agents from the storage and control module 130 and spraying the one or more materials or compositions (e.g. aerosol and/or disinfection agents) to the toilet bowl surrounding (e.g. toilet bowl surface, seat etc.).

In some cases, the external pipe 390 may rotate up/down or to the sides along an X-Y or X-Z axis of axis X-Y-Z to efficiently clean hidden locations at the toilets or the toilets surrounding. In some cases, as illustrated in FIG. 3G, the external pipe 390 is configured to rotate between 0-180 degrees in respect to axis Y along surface X-Y using one or more cogwheels 392 and 393 coupled to the motor 395. The motor 395 is configured to rotate the cogwheels 392 and 393 which in turn rotate the external arm 390 for spraying automatically one or more materials or compositions, such as disinfection materials to the toilet bowel cavity and/or specific locations according to one or more instructions received from the processor. In some cases, the external arm 390 may be rotated to the sides/up/down (e.g. 360 degrees) in respect to the treatment module while one or more materials such as sanitization materials are sprayed to the toilet or to the toilet surface.

In some cases, a spraying module 396 such as a sprinkler or spray nozzle may be coupled to the distal end of the external arm 390 for spraying the sanitization material upwards (e.g. in the Z axis direction) via one or more nozzles holes to the toilets bowel cavity or to the toilets surroundings.

In some cases, as illustrated in FIG. 3I the external arm 390 may be rotated and positioned perpendicular (e.g. between 60-90 degrees) to the treatment module 310 length, wherein the distal end of the external pipe including the nozzle spray is centered at the toilet bowl cavity for generating and spreading sanitization material 360 degrees (all directions) at the entire cavity or substantially the entire inner cavity and outer surroundings of the toilet bowel.

According to some embodiments as illustrated in FIG. 3H each nozzle may include a spraying head 381 including one or more openings 383 and 385. In some cases, each nozzle may be a compound nozzle in which several individual single or two fluid nozzles are incorporated into one nozzle body. This allows design control of drop size and sprays coverage angle. In accordance with other embodiments, another type of nozzles may be used.

Reference is now made to FIGS. 4A and 4B illustrating an isometric top and side views of a storage and control module 430, in accordance with embodiments. Typically, the storage and control module 430 is externally coupled to the outer side of the toilet bowl, however many other variations and adaptations may be used for placing the storage module 430, for example inside the toilet bowl or at other locations. The storage module 430 comprises a housing 410 having for example a first concave side cover 412 configured to be hanged in proximity to and in front of the outer side of the toilet bowel and a second compatible side cover 413 which may be convexly shaped. The housing 410 comprises a cavity including for example one or more containers which may be refilled automatically or manually with cleaning and disinfection materials in the form of liquid or powder or solid materials for cleaning and/or foaming and/or disinfecting the toilets.

For example, according to some embodiments, at the top or sides of the housing there are included one or more refill openings which may be sealable by one or more covers to seal the one or more inner containers. For example, the housing 410 may include two sealable openings 472 and 474 located at the top sides of the storage module 130 and may be covered by matching covers 472′ and 474′ which may be used to seal containers 478 and 479, for example by one or more clicking means.

In some cases, the device modules, such as the containers 478 and 479 may be easily releasable from the device and inserted back to the device. For example, the containers 478 and 479 may include a frame or other modules which can positively engage the body and/or frame to ensure that a suitable seal has been secured and may provide an audible or physical “click” type confirmation.

According to some embodiments, the device comprises one or more power buttons or switches, such as button 425 installed at the storage and control module 130, shaped for example as a round or square button configured to power the device 100 on and off. Typically, the device powers on when the button is pressed and powers off when the button is pressed again. In some cases, the device may include one or more sensors including voice recognition means for identifying a user command for activating the device on or off. In some cases, one or more buttons may be positioned on or connected to other modules such as the bridge 120 and/or the treatment module 110.

FIG. 4C shows an isometric view of the modules located inside the storage module 330, in accordance with embodiments. The storage and control module 330 comprise one or more pumps, such as first and second pumps 452 and 454, and a circuit board 432 such as a PCB including one or more processing modules such as processing module 431 and memory modules 433 and a power source 499. In some cases, the storage and control module 330 includes a single pipe.

The processing module 431 is configured to control the device module for automatically and autonomously cleaning toilets without any human intervention. In operation, the processing module receives data from the device modules including for example one or more indications received from the device modules such as the device's one or more sensors and process the data to yield output data for accordingly controlling the device modules.

In some cases, the processing module 431 is configured and enabled to receive movement or pressure indication from said sensors and operate the device spraying modules to spray fluids on the interior surface of a toilet bowl according to said sensors indications.

In some cases, the processing device may receive an indication of low cleaning material at the cleaning containers or at the cartridges and accordingly update the user to refill the containers or cartridge. In some cases the processing module may provide warnings or indications in the form of a “low material” or “low cartridge” status indicator light, or a series of flashing lights that require a user to consult the manual to decipher them.

In some cases, the processing module may launch a pop-up notification to a user computer screen or mobile device when cleaning toner levels are low.

In some cases, the device comprises a communication module configured to communicate to a server and other modules such as speakers to provide information on the module device status.

In some cases, the communication module is configured to transmit the collected data to a cloud-based server which is configured to analyze the data transmitted from the communication device, and a device configured to receive analysis results from the cloud-based server and present the analysis results to a user.

In some cases, the information includes the amount of cleaning materials in the device's containers; power level (e.g. low, high).

In some cases, the processing module may automatically and/or autonomously send instruction, for example by the communication module to refill the containers or to recharge the device.

The pumps, such as pumps 452 and 454 are configured to deliver respectively a cleaning and/or sterilization composition of a first type and a cleaning and/or sterilization composition of a second type. For example the cleaning or sterilization composition of the first type may be of perfuming composition, water coloring composition, non-stick composition, bleaching composition, foam generation composition, good filings and euphoria composition, pheromones, caustic composition and the composition of the second type may be or may include an alcohol composition for toilet's sanitization. The storage module 130 further comprises a case 497 comprising the one or more batteries 499.

In operation, the openings are refilled with cleaning chemicals or liquids which are further transmitted by transferring means such as pipes and/or pumps (e.g. pumps 452 and 454 and pipes 471 and 473) via the bridge module 420 into the treatment module which sprays the appropriate chemistry or cleaning fluids to the inner surface of the toilet bowl or to the toilets surroundings (toilet seat) to conduct the cleaning process.

In some cases, the cleaning materials (e.g. disinfection material) may be included in one or more dedicated refill cartridges which are configured to be positioned into the device containers, such as container 453 at the storage and control module 430. The cartridge may be detachably mountable for example to fixed contained within the storage and control module.

In some cases, the cartridges include one or more opening sealed for example by a sealing portion made of for example nylon. As illustrated in FIG. 4D, once the cartage is attached to the dedicated container 453, one or more pins such as pin 435 creates a hole at the cartridge and the cleaning material is pressed out of the cartridge and flows to the devices pumps via one or more pipes, and further to the sparing modules such as the nozzles and/or spraying arm for spraying the cleaning material to the toilets bowl cavity.

In some cases, the cleaning composition may be or may include chemicals materials configured and enabled to prevent infection. The composition may further include one or more of perfuming composition, water coloring composition, non-stick composition, bleaching composition, foam generation composition, good filings and euphoria composition, pheromones, caustic composition. The compositions may be in the form of one of: jell, tablets, liquid, powder or any other shape of materials as known in the art.

In some cases, the device may include materials and/modules for controlling the amount, color, intensity of the perfume or foam composition.

For example, the device may schedule according to predetermined intervals (e.g. by the processor module 431) the injection of the chemical martials as part of the toilet's treatment process. A treatment program may include for example a treatment schedule according to the following order:
a. softener material b. disinfections material c. whiting material d. anti-stick materials e. perfume materials f. coloring materials g. foamy materials.
   The treatment schedule may be in a different order or may include additional materials.

According to some embodiments, the chemical material composition is configured to provide a coloring reaction.

In some cases, the treatment chemical material is configured to slowly decompose, by using specific chemical materials controlling the chemical reaction.

In many embodiments, the device also includes a power source (e.g. one or more batteries 499) and one or more power source covers 498. In some embodiments, the device 100 is powered by a power supply from an external source. In some embodiments, the device 100 has an independent power supply.

In some embodiments, the batteries 499 may be placed within the storage module 130. For example, as shown in FIG. 4A the external cover 413 may include one or more movable battery covers 498 which can swing away or alternately may be replaceably removed with respect to the housing in order to provide access for a user to insert one or more batteries 499. The one or more batteries 499, may be one or more single use batteries or may be rechargeable batteries which provide a power supply to operate the treatment module 100.

In some embodiments, the storage and control module 430 may be connected to the bridge module 120 by connecting means such a holder 488 comprising a connection sleeve 489 configured to receive a portion of the bridge module. Although FIG. 4A shows a method performed by sliding a portion of the bridge 120 into a bridge holder 488, a person of ordinary skill in the art will recognize many other variations and adaptations for connecting the device 100 modules to one another.

According to some embodiments, the containers of the storage and control module, such as container 453 may have a volume of between 100-800 ml, for example 400 ml.

In some cases the length of the of the storage and control module 430 may be between 100-200 mm, for example 170 mm, the height between 50-150 mm for example 94 mm and the width between 30 mm to 100 mm, for example 65 mm.

In some cases the storage and control module 430 may include a container or housing F for holding the power source 499.

Reference is now made to FIGS. 5A and 5B illustrate isometric upper side views of a bridge module 520, in accordance with embodiments. The bridge module 520, in some cases, is shaped as an elongated flexible strip which may be hanged along the surface of a section 225 of the toilet bowl rim 240 and hold the treatment module 110 and storage and control module 130, of device 100 or device 150 or other cleaning devices illustrated herein for example at the sides and below the toilet bowl rim 240. The bridge module 520 comprises one or more pipes (e.g. pipes 473 and 471) attached for example to the bridge module 520 surface for delivering and supplying cleaning materials (e.g. aerosol and other agents) and/or fluids to the spraying modules such as the first sparing module 360 and/or external arm 390.

In some cases, the pipes may be made of plastic or other materials as known in the art.

Alternatively or in addition to the pipes the device 100 may include one or more channels or pipes formed internally as part of the device inner modules for supplying treatment materials and/or fluids to the spraying module 360 or 390. In some cases the materials may be delivered by air pressure means.

Specifically, the bridge 520 comprises a flat stripe section 522, preferably configured to accommodate the geometries of different types of toilet bowls, and two flexible sections extending from both ends of the flat stripe sections configured to be folded along the inner and external sides of the toilet bowl rim 240. For example, a first flexible section 523 extending from section 522 may be bended in a perpendicular or substantially in perpendicular (e.g. between 60-90 degrees) in respect to the toilet bowl rim surface downwardly towards the outer side of the toilet bowl.

According to some embodiments, the distal end 510 of the first flexible section 523 is configured and enabled to slide into the bridge holder 488 and hold the storage module 130 at the external side and below the toilet bowl rim. It is noted that other methods and configurations may be used to connect the bridge module 520 to the external and/or the treatment module.

The bridge module 520 further comprises a second curved section 524 extending from the distal end of the flat stripe section 522 downwardly towards the interior of the toilet bowl. The second section 524 is configured and enabled to be connected to the treatment module and hold the treatment module below the toilet bowl rim 240.

According to some configurations, the bridge module 520 may include a second spraying module 590 (e.g. upper spraying module) such as an aerosol module for cleaning and disinfecting the toilet bowl. The second spraying module 590 may comprise one or more spraying devices such as nozzles, such as nozzle 592 connected via one or more pipes to the storage module 130 and configured and enabled to spray aerosol into the bowel interior surface.

In some embodiments, the second spraying module 590 may be positioned at the distal end of the second curved section 524 so as to enable direct spraying to the upper surface of the toilet bowl, for example to the toilet bowl areas which may not be sprayed by nozzles 363, 366 and 369. As illustrated in FIGS. 2B and 2C the second spraying module is configured and enabled to spray cleaning materials (such as streams 244) and/or disinfection material via a plurality of nozzle on a toilet seat 250 or toilet space.

In some cases, the bridge module 520 may include one or more indicator modules, such as indicator module positioned at the outer surface of the upper portion 525. The indicator module may include one or more lights such as LEDs 593, 594 indicating the status of the system (e.g. on or off) or which module (e.g. upper or lower spraying modules) is currently activated.

According to some embodiments, the bridge module 520 comprises or is connected to electrical generator means for example a hydropower module 540 for producing hydroelectricity to, for example, charge the device's power source, e.g. batteries 499 and thus provide power to activate the device's electronic modules for example LEDs 593, 594. The hydropower module 540 comprises one or more rotating coils of wire such as water wheels to convert mechanical rotation into a pulsing electric current. For example, according to some configurations, the second strip section 524 comprises a ‘T’ shaped portion 526 curving away between 30-60 degrees in respect to the upper portion 525 of the bridge. The ‘T’ shaped portion 526 comprises two arms 542 and 544 extending to the sides from the second strip section 524 and configured to hold one or more electrical generator wheels, such as wheels 545 using respectively pins 569. According to one configuration, as illustrated in FIG. 5C, portion 526 may be attached to the sides of opening 320 of the treatment module 112 and the wheels 545 may partially or completely be inserted into the treatment module 112 cavity.

FIG. 5D shows a side view of the water wheel 545, in accordance with embodiments. The water wheel 545 may be octagonally shaped comprising a pivot 561, a number of cavities 566 and blades 567 which are mounted around the circumferential rim of the wheel to prevent the escape of fluids from the ends of the cavities 566 until they have moved a certain distance.

In operation, toilet's flushing fluids are directed via the opening 320 of the treatment module 112 to the blades of the water wheels, creating a force on the blades. In this way, energy is transferred from the water flow to the wheels (e.g. turbine) producing hydroelectricity in the way water turbine (e.g. Pelton wheel) is operated.

Alternatively or in combination, the wheels, such as wheels 545 may be used as a meter such as a measurement module for measuring the toilet flush water speed or status (e.g. flushing time) for indicating if the toilet use was completed and the type of use according to the flushing duration. The indications such as the toilet flushing speed and time may be transmitted to the one or more processing module for activating accordingly the suitable cleaning process. For example, an indication of a short use of the toilets may activate a first cleaning process by for example the cleaning module 360 for a short period, while an indication of a long use of the toilets (e.g. as a result of indication of flushing for a long period) will automatically activate the rotatable cleaning arm for sanitizing the toilets and/or the cleaning module for a longer time.

Alternatively or in combination, the device may include other energy generators and motors by way of non-limiting examples these include one or more of: a stepping motor, air pressure means, spring pressure means, chemical reaction mans, mechanical or electrical pressure amplifiers rotary motor or any motor which is configured to activate the system cleaning modules such as the system's nozzles or sensors.

According to some embodiment, the bridge module 520 dimensions may be between 50-130 mm long such as 100 mm at the center section (i.e. the flat stripe section 522) and may extend between 50-100 mm, for example 90 mm at one side (i.e. the second section 524) and between 50-100 mm, for example 60 mm at the other extending section (i.e. section 523). In some cases, the bridge width (i.e. section 522) is around 30-50 mm for example 40 mm.

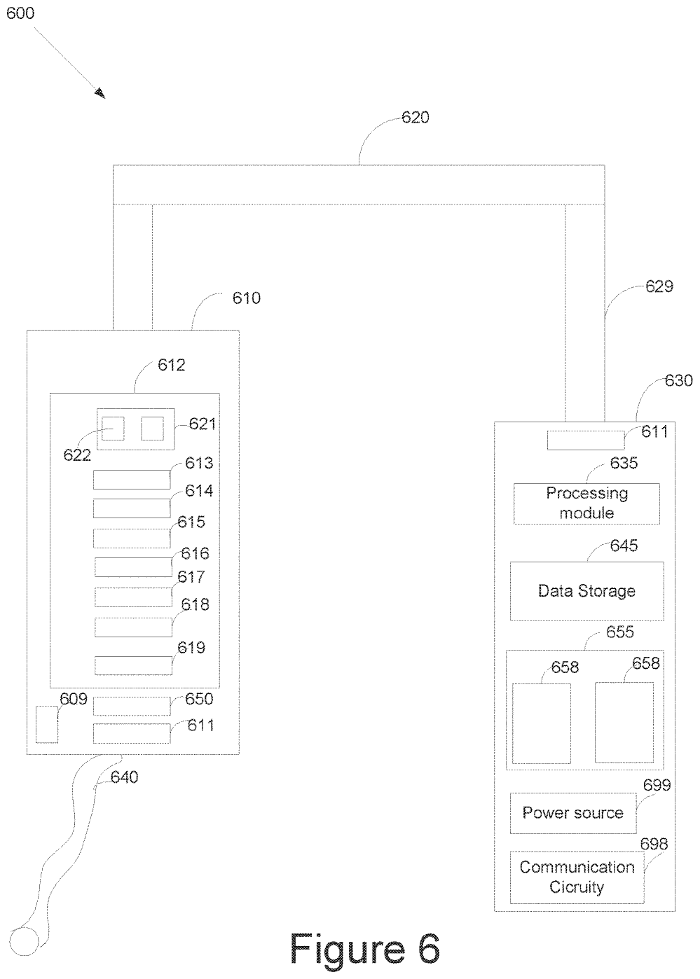

Reference is made to FIG. 6 illustrates a block diagram side view of a device 100 configured to be placed in toilets compartments for detecting and identifying substances such as explosive or toxic substances, infectors, drugs, microbes such as infectious microbes and the like, in accordance with embodiments. In some cases, the device 600 may be connected or may be included in a toilet cleaning device as known in the art, such as a toilet dispenser or a device as illustrated in U.S. Provisional application 62/514,854 and PCT application number PCT/IL2018/050601 which incorporated herein by reference.

According to some embodiments, the device 600 may include a first module such as a bridge module (e.g. suspension module 620) configured and enabled to be hanged for example on a toilet bowl rim and hold one or more modules such as a second module (e.g. treatment module 610) and a third module (e.g. external module 630).

In some cases, the bridge module 620 includes a spraying module configured to spray cleaning materials via a plurality of nuzzles on a toilet seat or toilet space.

In some cases, the treatment module 610 may include a housing module which may be connected to the a distal end of the bridge module 620 and positioned away from the toilet bowl, for example below the toilet boil rim at the path of the flushing water and the external module 630 may include a housing configured to be connected to the opposite distal end of the bridge module 620 and may be positioned externally to the toilet boil. The external module 630 may comprise a housing having for example a first concave side cover configured to be hanged in proximity to and in front of the outer side of the toilet bowel and a second compatible side cover which may be convexly shaped.

The treatment module 610 may include one or more modules for cleaning the toilet bowl such a one or more containers 609, one or more spraying modules 611 for dispersing cleaning composition and/or fluids such as flushing tank fluids into the toilet bowl.

The one or more containers 609 are configured and enabled to collect and store fluids, such as fluids toilet flushing tank fluids

The treatment module 610 may further include a cleaning module 650 and a sensing module 612. The cleaning module may include one or more spraying modules (e.g. second spraying module) configured to spray fluids and cleaning materials via a plurality of nuzzles into a toilet bowl. According to some embodiments, the sensing module 612 includes one or more sensors configured to detect and identify substances such as chemical/biological/radiological/nuclear/explosive agent, drugs, or toxic materials or the like. In some cases, the detection is performed in real time for allowing immediate identification of the risk and the saboteur which used or will use the agent.

In some cases, the sensing module 612 may include a sensor array for detecting infectors, drugs, diseases such as diabetes, cancer, microbes such as infectious microbes or toxic compounds. For example the array may include different types of sensors, such as one or more sensors 613 for detecting explosive agents and/or one or more sensors 614 for detecting weapons or weapon substances, and/or one or more sensors 615 for detecting radioactive materials or radioactive indication substances and/or one or more sensors 616 for detecting diseases agents such as diabetes, cancer etc., and/or infectors, drugs, microbes such as infectious microbes, and/or one or more sensors 617 for detecting drugs and/or one or more sensors 118 for detecting alcohol and/or one or more sensors 619 for detecting and analyzing DNA macromolecules.

According to some embodiments, the sensor module 612 (e.g. sensors 613-619) is configured to receive or collect air, liquid or solid samples found in toilet waste such as feces and urine. For example, toilet waste mixed with toilet's flushing water is sampled by the sensor module 612. According to some embodiments, the samples are collected and received at the sensors by one or more tubules 640 or pipe-like modules which are attached to the sensors at a first end and to the inner surface of the toilet bowl for example to the interior toilet bowl perimeter to efficiently receive samples of a toilet user defecation. In some cases, the samples are received using suction methods, e.g. vacuum etc.

In some cases, the sensor module 612 may include an imaging module 621 comprising one or more imagers such as high-resolution imagers 622.

In some cases, the external module 630 may include a processing module 635, data storage module 645, a cavity 655 including for example one or more containers 658 which may be refilled automatically or manually with liquid or power chemicals for cleaning and/or foaming and/or disinfecting the toilet. The processing module 635 may be connected or may be in communication with the sensor module 612 and imaging module 621 may include one or more processors for analyzing the sensed samples based on various data analysis algorithms for generating diagnostic data results including for example the type, amount, location of afflicted substances found within the samples. The diagnostic data may be stored at the storage module 645 or at the remote server storage. The second housing 630 is configured to store cleaning materials or compositions such as deodorizing and disinfecting materials (e.g. detergents) which may be dispensed by the treatment module 650 into the toilet bowel. For example, according to some embodiments, at the top or sides of the housing there are included one or more refill openings which may be sealable by one or more covers to seal the one or more inner containers.

The external module 630 may further include one or more pumps, such as a first pump for delivering a cleaning or sterilization composition of a first type and a second pump for delivering a cleaning or sterilization composition of a second type. For example, the cleaning or sterilization composition of the first type may be of perfuming composition, water coloring composition, non-stick composition, bleaching composition, foam generation composition, good filings and euphoria composition, pheromones, caustic composition and the composition of the second type may be or may include an alcohol composition for toilet's sanitization. In operation, the second module openings are refilled with cleaning chemicals or liquids which are further transmitted by transferring means such as pipes and/or pumps via the bridge module into the treatment module which sprays the appropriate chemistry or cleaning fluids to the inner surface of the toilet bowl or to the toilets surroundings (toilet seat) to conduct the cleaning process.

In some cases, the cleaning composition may be or may include chemicals materials configured and enabled to prevent infection. The composition may further include one or more of perfuming composition, water coloring composition, non-stick composition, bleaching composition, foam generation composition, good filings and euphoria composition, pheromones, caustic composition. The compositions may be in the form of one of: jell, tablets, liquid, powder or any other shape of materials as known in the art.

In some cases, device 600 may include materials and/modules for controlling the amount, color, intensity of the perfume or foam composition.

In many embodiments, device 600 also includes a power source module 699 including one or more batteries. In some embodiments, the device 600 is powered by a power supply of an external source. In some embodiments, the device 600 has an independent power supply.

In some embodiments, power source 699 may be placed within module 630.

In some embodiments, the device 600 may include a wireless communication circuitry 698 to couple to the device, for example within housing 630, and communicate with a remote server. The wireless communication circuitry 698 may include one or more receivers and transmitters and/or transceivers for receiving/transmitting data (e.g. captured images and/or sensory data) to a remote module such as a remote server.

In some cases, each of the device 600 modules may include or may be connected to the wireless communication circuitry for receiving and/or transmitting data from the device's 100 modules or from external modules such as network based cameras or other detecting or communication means.

In some embodiments, the external module 630 may be connected to the bridge module 620 by connecting means such a holder comprising a connection sleeve configured to receive a portion of the bridge module.

In some cases, the bridge 620 comprises one or more indication modules such as warning and status lights or small speakers to indicate the device mode, e.g. sleep/active mode etc.

In some cases, a gasket, O-ring, adhesive seal, or other water-repellant sealing means known in the art may be used to seal the device 600 and/or modules within the device such as the sensing module 612.

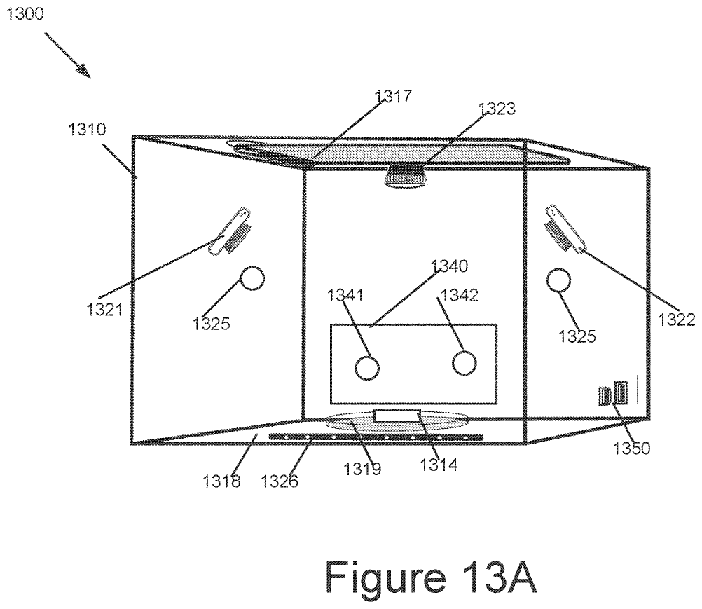

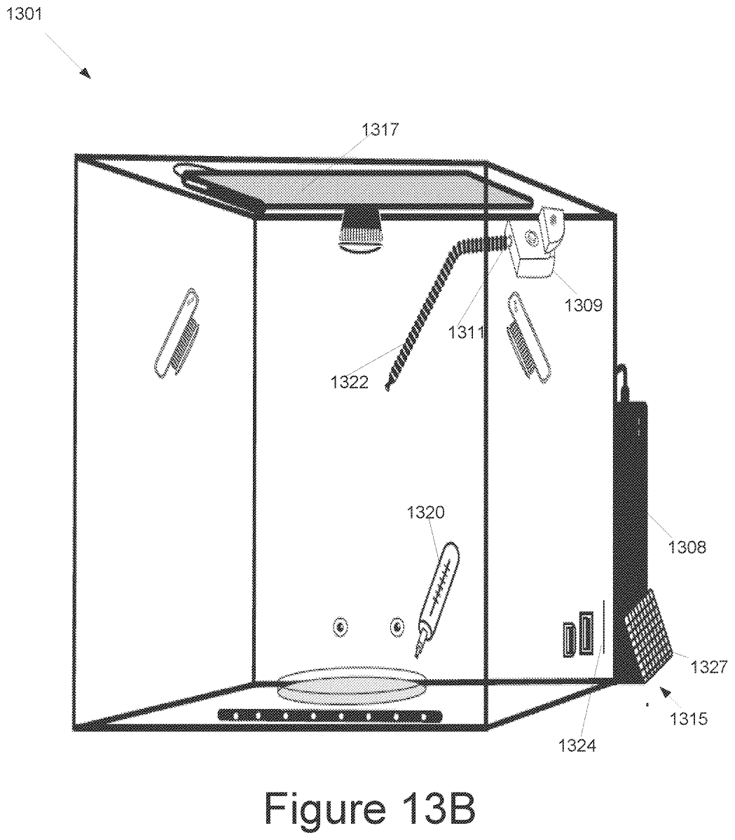

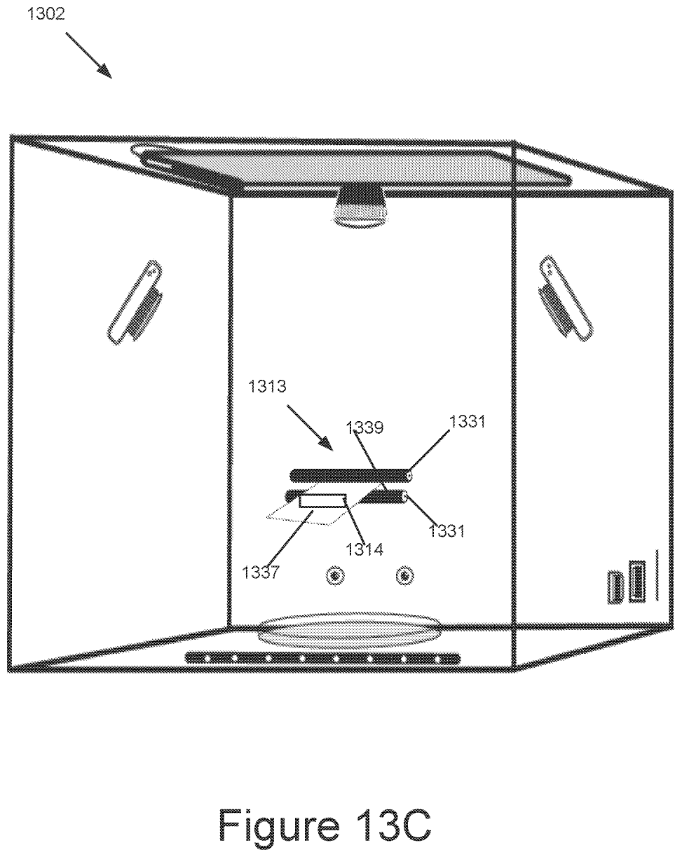

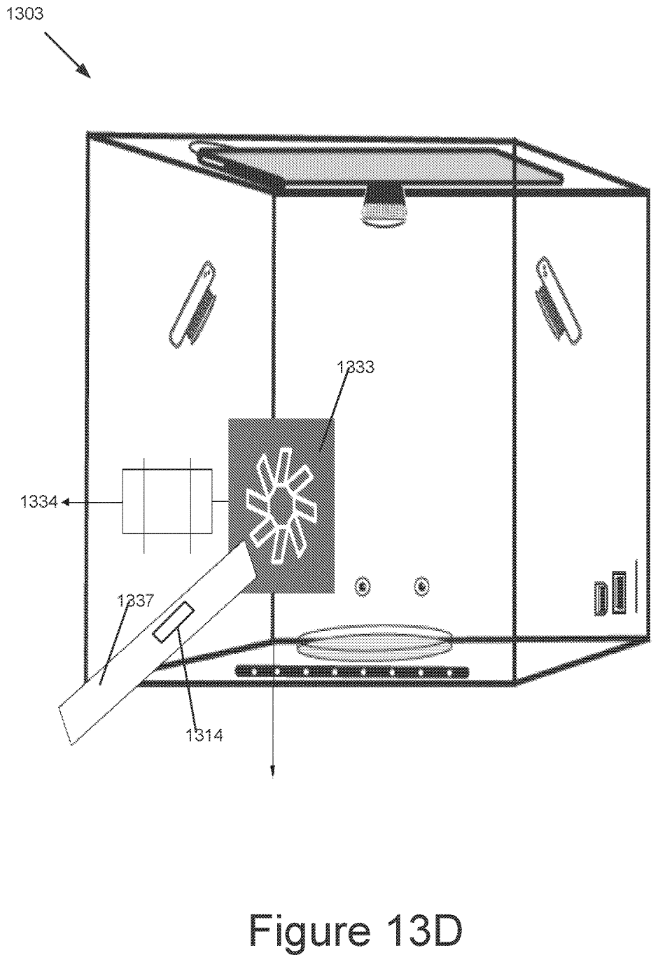

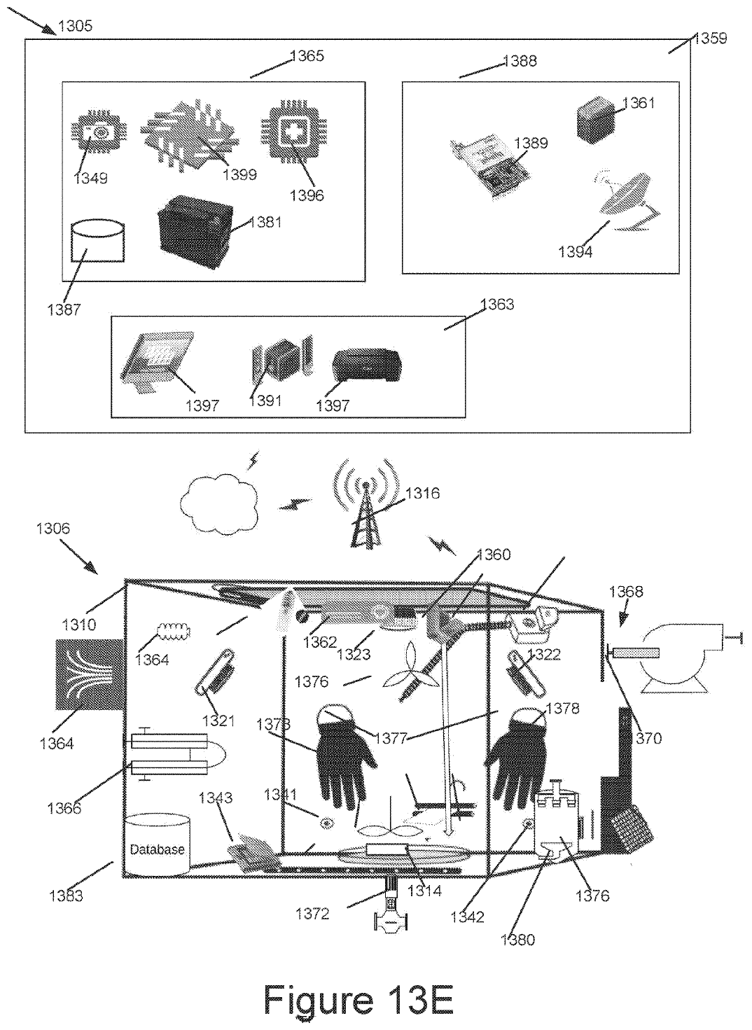

In some cases, the sensing module 612 and the imaging module 621 and the container may be included in a housing such as the housing 1310 of FIGS. 13A-13E. In some cases, the housing may be included in the external module 630.

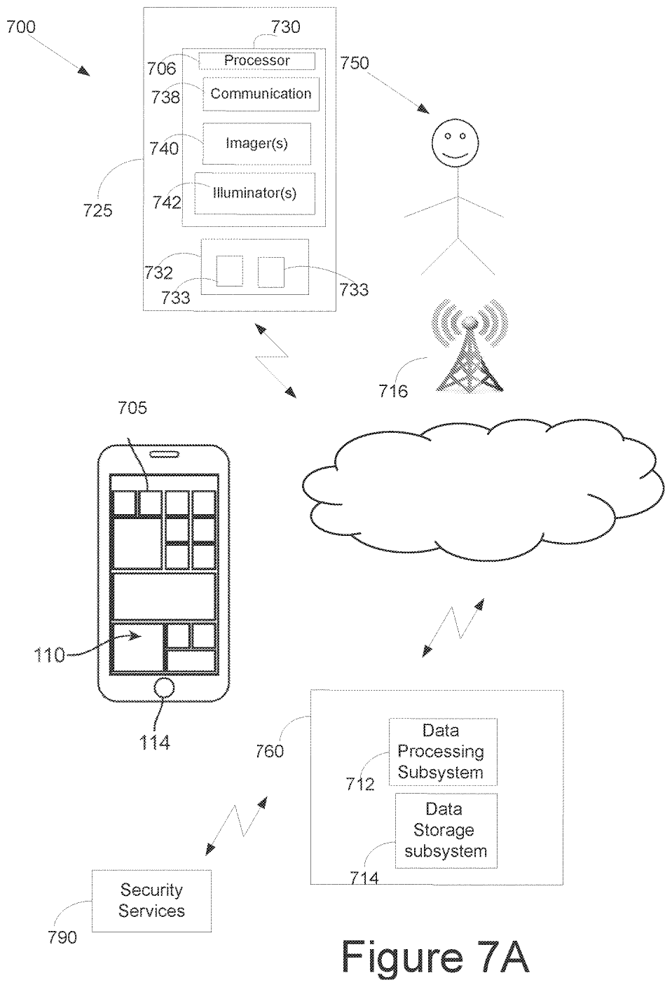

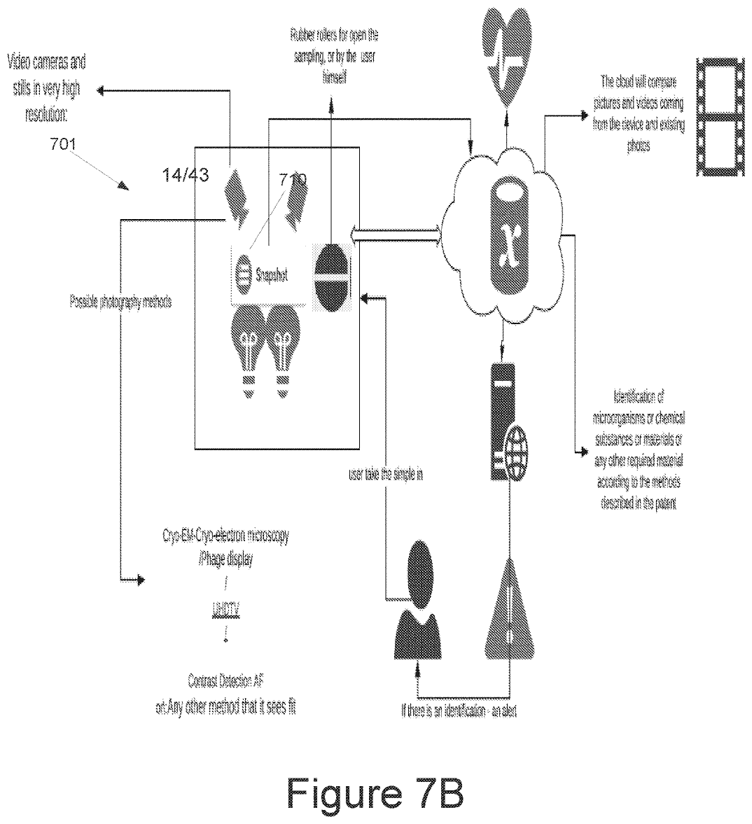

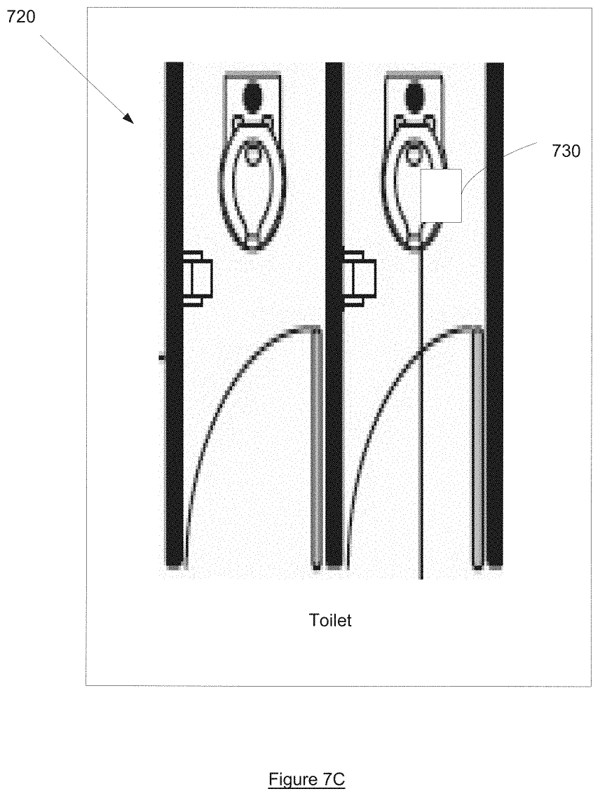

FIG. 7A illustrates a block diagram of a system 700 such as a network system for detecting, diagnosing and alerting on substances such as chemical-biological-explosive (CBE) or other substances which may be or may include for example threats on-site and allow for immediate responders to mitigate spread, risk, and loss, in accordance with embodiments. The system 700 comprises a device 730 which may be in wireless communication 716 with a cloud based remote server system 760 comprising a communication circuity 738, the data storage subsystem 714 and data processing subsystem 712. In some cases, the device 730 may also be in wireless communication 716 with a hand held device 705 for transmitting alerts to security services 790. In some cases, the hand held device may include data storage subsystems and/or data processing subsystems.

The device 730 is configured and enabled to image one or more substances 733 such as chemical-biological-explosive (CBE) or other substances in a scene, for example in toilets compartments, or other places. Specifically, in some cases, the device 730 may be attached to a toilet bowel rim or may be placed in a toilet cleaning device. For example, the device 730 may be part or may be included in a toilet cleaning device such as the cleaning device shown in FIGS. 1-6.

According to one embodiment, the communication circuitry 738, imager(s) 740 and the illuminator(s) may be part of the device 730 or the portable device 705 or other devices such as the toilet cleaning device. Alternatively, the device 730 may be included within a housing 725 such as a case or a jacket configured to be releasable (i.e. connected or disconnected) to one or more devices such as the portable device 705. For example, the device 730 may include the imager(s) and illuminator(s) and the communication circuitry 738 may be part of the housing 725 which is electrically or wirelessly connected to the portable device 705 or other devices such as a cleaning device, for example through a dedicated connection such a USB connection, wireless connection or any connection known in the art.

According to one embodiment, the housing 725 may further include a housing 732 for holding one or more substances 733 or samples (e.g. OUT) of the substances to be imaged by the imagers 740 and the illuminators 742 and diagnosed by the data processing subsystem.

According to one embodiment, the device 730 may be in wireless communication 716 with a cloud based server 760 comprising a data storage system 714, including for example a universal database, by a wireless communication circuity, included in the imaging module 730 and device 710. The universal database may be operated by a cloud server, where the diagnosed data associated with sample materials may be stored. The sensing device 710 can acquire the data as described herein. The device 710 may comprise a processor and communication circuitry as described herein. The sensing device 710 can acquire the diagnostic data as described herein while the imagers acquire images of suspects and may transmit the data to the cloud based storage subsystem 714. The data can be processed and analyzed by the cloud based server 760, and transmitted to one or more security services 790 for updating and activating police services for capturing the identified suspects. In some cases, the cloud based system or server 760 may be accessed remotely, for example via a wireless internet connection, by one or more hand held devices such as mobile phones (e.g. smart phones) of security authorities. In many embodiments, the cloud server is simultaneously accessible by more than one users/hand held devices of the system.

In accordance with embodiments, system 700 may allow multiple sensing devices and imaging modules to connect to the cloud based server 718, as described in further detail herein. The ability of the system to support a large number of users and devices at the same time can allow users of the system to access, in some embodiments in real-time, large amounts of information relating to a suspected person, such as a terrorist group. Access to such information may provide security forces and general authorities such as airport security with a way of making informed decisions relating to a material of interest received.

The device 725 may comprise a substantially stationary device when used, such as a wireless communication gateway, for example. The device 710 and/or the server may comprise a processor (e.g. data processing subsystem 712 or processor 706). The processors 706 or 712 may comprise a tangible medium embodying instructions, such as a computer readable memory embodying instructions of a computer program. Alternatively or in combination the processors may comprise logic such as gate array logic in order to perform one or more logic steps. The device may comprise a memory with a database of data stored therein, and a processor with analysis software programmed with instructions. The memory can be volatile or non-volatile in order to store the measurements in the memory.

An analyzed substance can determine whether a mixture comprising the substance being investigated contains cells associated with components. The components can, for example, be a substance, mixture of substances, or microorganisms such as infectors, drugs, diseases such as diabetes, cancer, microbes such as infectious microbes or toxic compounds. Non-limiting examples of such substances include toxins, decomposition products, or harmful microorganisms.

Because of its small size and low complexity, the device herein disclosed can be integrated into or is connectable to a toilet cleaning device such as a toilet dispenser or cleaning device. It can either be enclosed within the cleaning device itself, or mounted on the device and connected to it by wired or wireless means for providing power and a data link. By incorporating the sensing device into a cleaning device, the sensing data obtained can be uploaded to a remote location, analysis can be performed there, and the user notified of the results of the analysis. The device can also be equipped with a GPS device and/or altimeter so that the location of the sample being measured can be reported.

FIG. 7A illustrates a block diagram of a system 701 such as a network system for detecting, diagnosing and alerting on substances such as chemical-biological-explosive (CBE) or other substances which may be or may include for example threats on-site and allow for immediate responders to mitigate spread, risk, and loss, in accordance with another embodiment.

In some cases, as illustrated in FIG. 7C the device 730 may be posited in a scene, for example the imagers 740 may be positioned in proximity to the toilet compartments 720, for example at the vicinity of the toilet entrance or at other locations for monitoring and/or imaging one or more substances, for example in real time.

According to some embodiments, the device may be fixed to the toilet bowel inner surface. Consequently, the sensor device may be removed and repaired, cleaned, or replaced if damaged or fouled.

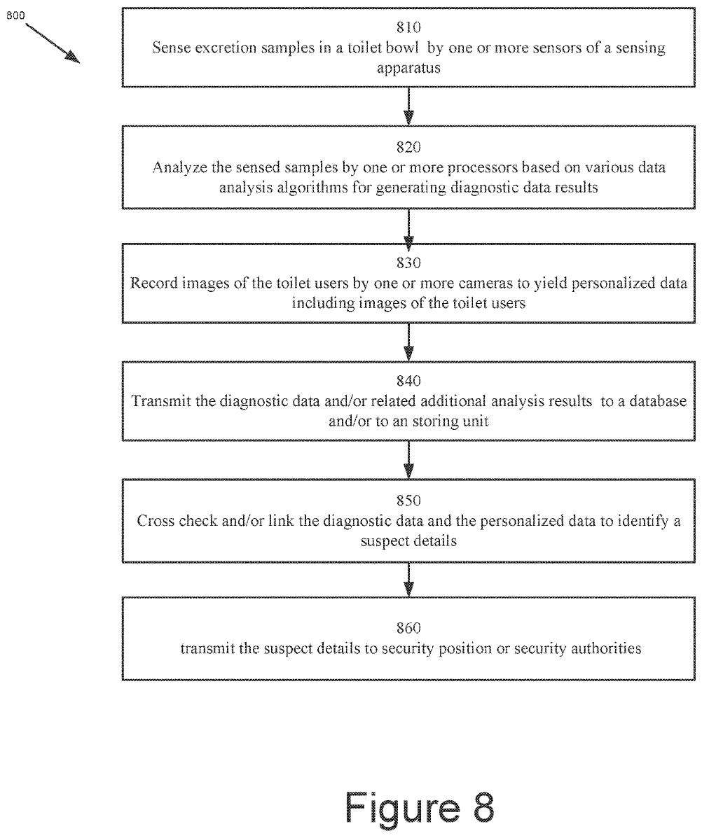

FIG. 8 is a flowchart 800 of a method for detecting diagnosing and alerting on substances such as chemical-biological-explosive (CBE) or other substances threats on-site and allow for immediate responders to mitigate spread, risk, and loss, in accordance with embodiments. At step 810 one or more substances or objects such as excretion samples in a toilet bowl are sensed by one or more sensors of a sensing device such as a device 600 or devices 1300-1306. At step 820 the sensed samples are analyzed by one or more processors based on various data analysis algorithms for generating diagnostic data results including for example the type, amount, location of afflicted substances found within the samples. At step 830 recordings such as video or images recordings of the toilet users are recorded by one or more cameras to yield personalized data including images of the toilet users including for example, user image, time stamp, location etc. For example, the images may include video recordings of users entering or departing to or from one or more predefined zones such as an airport entrance, a university etc. At step 840 the diagnostic data and images and related additional analysis results may be transmitted to a database and/or to a storing module including memory and/or processor such as storage module 718 and/or processor 735 of remote server 260.

In some cases, once a threat or a hazard is alerted, for example by the devices 600 or 1300-1306 due to hazardous substances found in the toilet, the device initiates a request to image users located in the toilet's vicinity, and/or to track suspects (e.g. based for example on face recognition algorithms).

At step 850 the diagnostic data and the personalized data are cross checked and/or linked to identify and verify the suspect details (e.g. which the diagnosed affected samples relate to) to provide the suspect's detail file including: image, name, age, gender, the type of threat, location, time etc. At step 360 the suspect details are transmitted to security position and/or to security authorities.

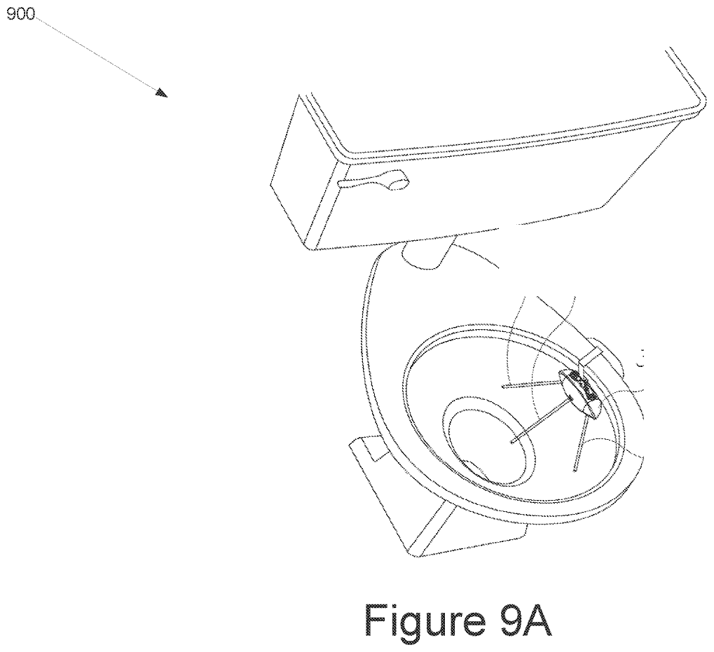

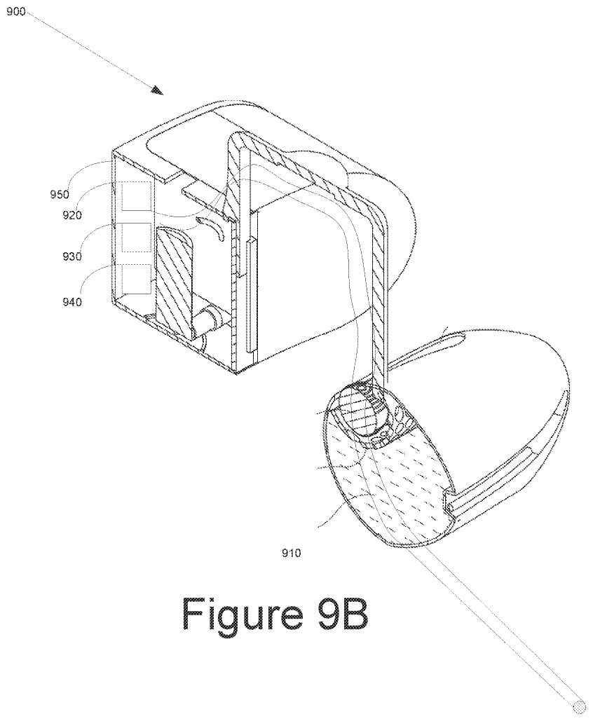

Reference is now made to FIG. 9A illustrates an isometric top view of a cleaning and detection device 900. In accordance with embodiments, the device 900 is configured to detect and diagnose substances such as infectors, drugs or toxic compounds and more particularly, to detect and alert of threats found in the toilet or toilets compartments such as infectors explosives drugs or toxic compounds.

In accordance with some embodiments as illustrated in FIG. 9B the device 900 may include one or more tubules or small knitting needle such as tubule 910; and/or one or more image sensors such as imagers 920; and/or one or more chemical reaction or chemical reagents 930 such as color reagents or color reaction which are used to transform colorless chemical compounds into colored derivatives which can be detected visually or with the aid of a colorimeter; and/or one or more sensors such as an sensor array 940.

According to some embodiments, the imagers 920 and/or the chemical reaction or chemical reagents 930 an/or the sensor array may be included in a chamber 950 configured to receive the sample from the tubule 910 via one or more openings.

The tubule 910 may include an elongated tube which is configured to be in contact with the toilets fluids for obtaining samples from toilet waste, for example mixed with toilet's flushing water. For example, as illustrated in FIG. 9B, one or more tubules 910 may be attached at one side to the treatment module and the other side of the tubules 910 may reach the toilet bowl surface. In operation, the toilet waste is sampled and are sucked into the treatment module, for example via one or more openings for analyzing the obtained samples using one or more sensors such as chemical or biological sensors. The analysis may be further processed by a processing module or by an external processing module. The analysis results may be further transmitted to an external device for example via the communication module or to a server based processor or memory.

According to some embodiments, the imagers 920 are configured to generate one or more images of the toilet such as the toilet bowel interior surface and/or toilet seat and/or toilet surroundings. The obtained images are transmitted, for example wirelessly to the processing module or to an external processing module such as a mobile device (mobile phone) processing module for analyzing the received images. The analysis may include comparing images to one another or to other predefined images, such as images stored at a memory server or social networking websites to detect suspected change found in the obtained images and for further diagnosing of substances such as infectors, drugs or toxic compounds and the like.

According to some embodiments, the imagers may be CCD images or CMOS images or any imagers as known in the art. In some cases, the imagers are configured to generate high quality images and may include lenses in order to magnify the objects.

In some cases, the tubule 910 may include one or more chemical reagents or sensors. In operation, the sensor within the tubule may identify suspected materials found within the toilet bowl. At the next step a sample is obtained by the tubule. The sample is diagnosed by one or more sensors such as the sensor array or chemical or biological reagents and may be further compared with obtained images of the obtained sample. In some cases, the images are transmitted via the communication module and/or IoT (Internet of Things) means for analyzing and comparing the sample to yield information on the obtained sample, for example to identify infectors, drugs, diseases such as diabetes, cancer, microbes such as infectious microbes or toxic compounds.

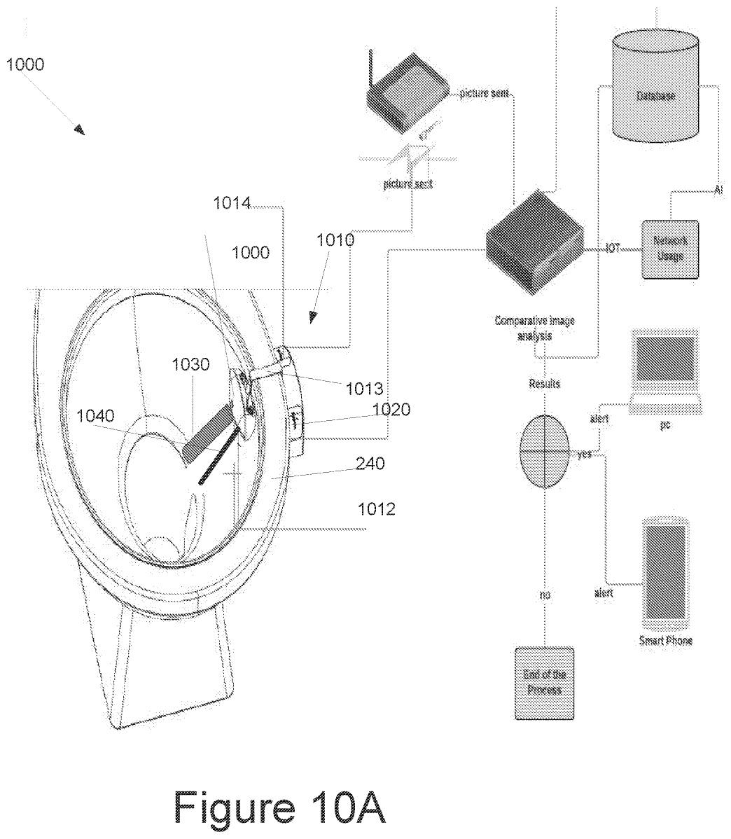

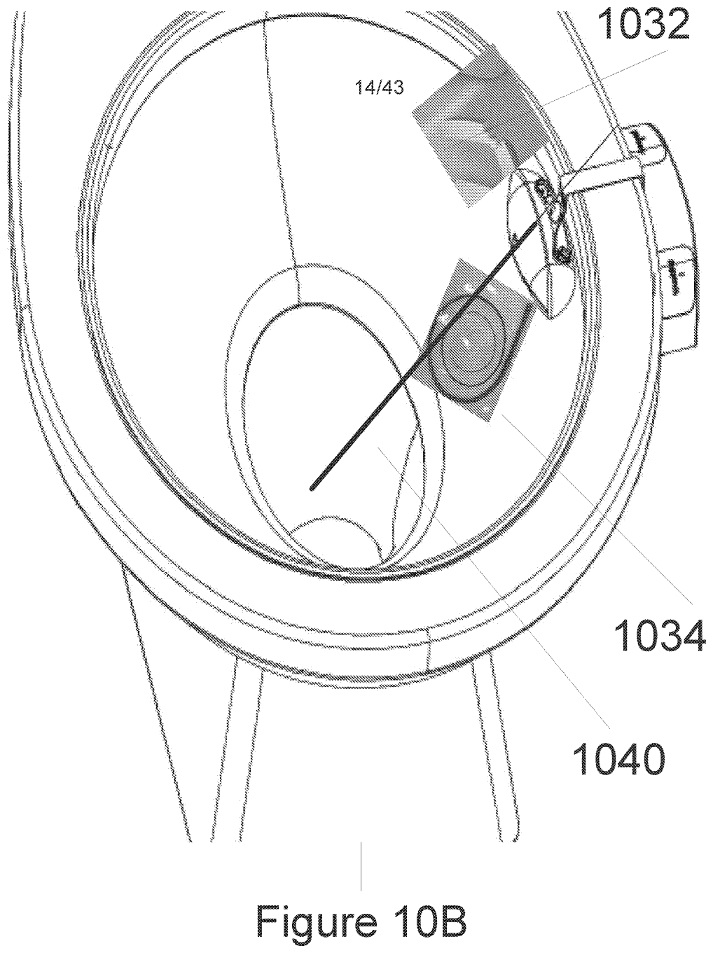

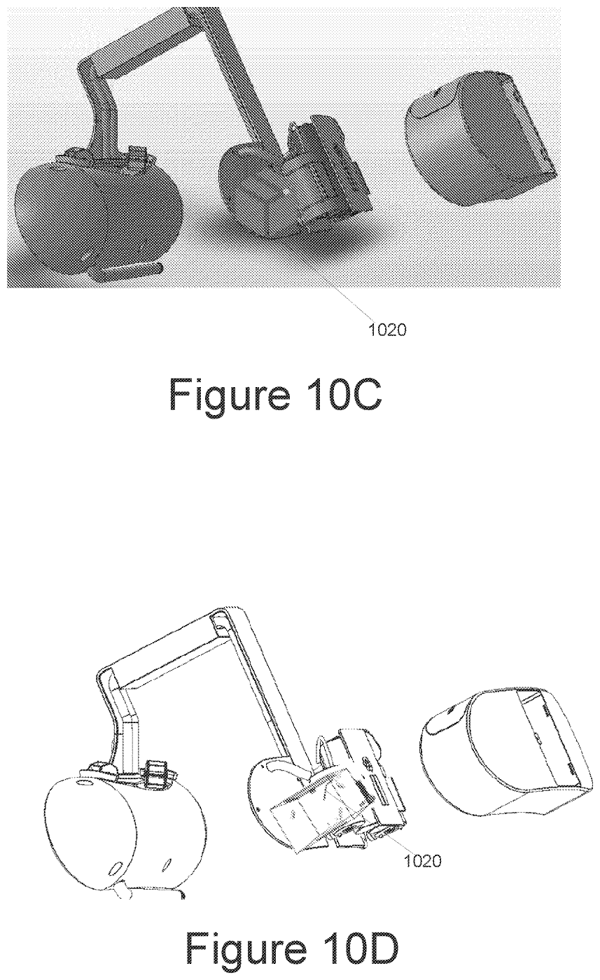

FIG. 10A illustrates a block diagram of a system 1000 and a top side isometric view of a device 1010, hanged on a toilet bowl rim 240, wherein the system 1000 and device 1010 are configured to detect and/or identify and/or diagnose an OUT such as suspected materials or substances found within the toilet bowl or the toilet surrounding, in accordance with embodiments. In some cases, the OUT may be or may include chemical-biological-explosive (CBE) or other substances and the system 1000 may automatically report on these threats or on or other threats on-site and allow for immediate responders to mitigate spread, risk, and loss, in accordance with embodiments. Specifically, the device 1010 is configured to receive or collect air, liquid or solid samples found in toilet waste such as feces and urine, e.g. toilet waste mixed with toilet's flushing water.

According to one embodiment, the device 1010 may be for example the cleaning device 100 or 150 illustrated in FIGS. 1A-1G and 3A-3I and may include one or more of the device's 100 and 150 modules and elements. For example, the device 1010 may include a treatment module 1012, a bridge module 1013 and an external module 1014. In some cases, the treatment module 1012, the bridge module 1013 and the external module 1014 may be or may include some or all the elements of the treatment module 610, bridge module 620 and external module 620 of FIG. 6 or the treatment module, bridge module and external module illustrated in FIGS. 1A-1G and 3A-3I. Device 1010 may further comprise a collecting module 1030 for collecting or receiving the OUT from the toilet and a diagnosing module 1020 for characterizing and/or diagnosing the collected OUT. In some cases, the collecting module may be connected externally to the treatment module 1012 positioned away from the toilet bowl, for example below the toilet boil rim at the path of the flushing water. In other embodiments, the collecting module may be connected to other sections of the device 1010. In some cases, as illustrated in FIGS. 10C and 10D the diagnosing module 1020 may be included in the external module housing. In accordance with an embodiment, the diagnosing device size may be in the range of 2×2×2−1×1×1 cm³ to few microns or more. For example, less then 1×1×1 cm³. In some cases, the size may be more then 2×2×2 cm³. According to some embodiments, the diagnosing module 1020 may be or may include one of devices 1300, 1301, 1302 and 1303 illustrated in FIGS. 13A-13E, in accordance with embodiments.

In operation, the collecting module 1030 collects samples (OUT) from within the toilet bowl and delivers the collected OUT into the treatment module 1012 which further transfers the OUT via one or more pipes to the device 1020 which is configured to capture one or more images of the OUT in high resolution mode and locally diagnose the OUT using one or more processors and/or transmit the captured images to a remote server comprising external processor and database configured to compare the captured images to one or more existing images or data stored at the database and identify the OUT. In some cases, the identification includes characterizing the OUT to identify it's content (size, material, amount, type etc.) and may automatically report on these threats or on or other threats on-site based on the findings and allow for immediate responders to mitigate spread, risk, and loss, in accordance with embodiments.

In some cases, the substances may be inserted to the device manually by a user. In some cases, the diagnosing may be released or repaired or exchanged from the device manually.

According to some embodiments, as illustrated in FIG. 10B, the collecting module 1030 may be selected from the group consisting of: one or more mechanical arms 1032 such as robotic arms; tubules for collecting and/or sucking samples found in toilet waste (OUT); whip type element 1034 made of for example elongated sticky and flexible material which is configured to preform whiplash movement and stick one or more samples within the toilet to it's end and insert them back into the device 1010, pump, vacuum apparatus, pincer, conveyor, a hook. In some cases, the whip may be made of plastic or any type of sticky material. In some cases, the tubules may include or may be attached to one or more arms such as the robotic arm 1032 which are configured to automatically and/or autonomously collect the OUT found in the toilet. In some cases, the robotic arm may include one or more imagers for imaging the OUT.

According to some embodiments, the device 1010 may include an optical fiber 1040 comprising one or more imagers (for example at the optical fiber distal end which is within the toilet bowel) configured to capture one or more images of the OUT (samples within the toilet) and transmit the captured images to the device 1010 and/or to an external device via a remote server comprising a processor for analyzing the captured images as described herein above. In some cases, the imagers are located at the device, for example inside the device 1000 or external to the device to image the substances once they are inserted into the device 1000. In accordance with embodiments, the imagers may in the size of several microns and are configured to be inserted to a fluid surrounding, such as the toilet's fluid (e.g. water).

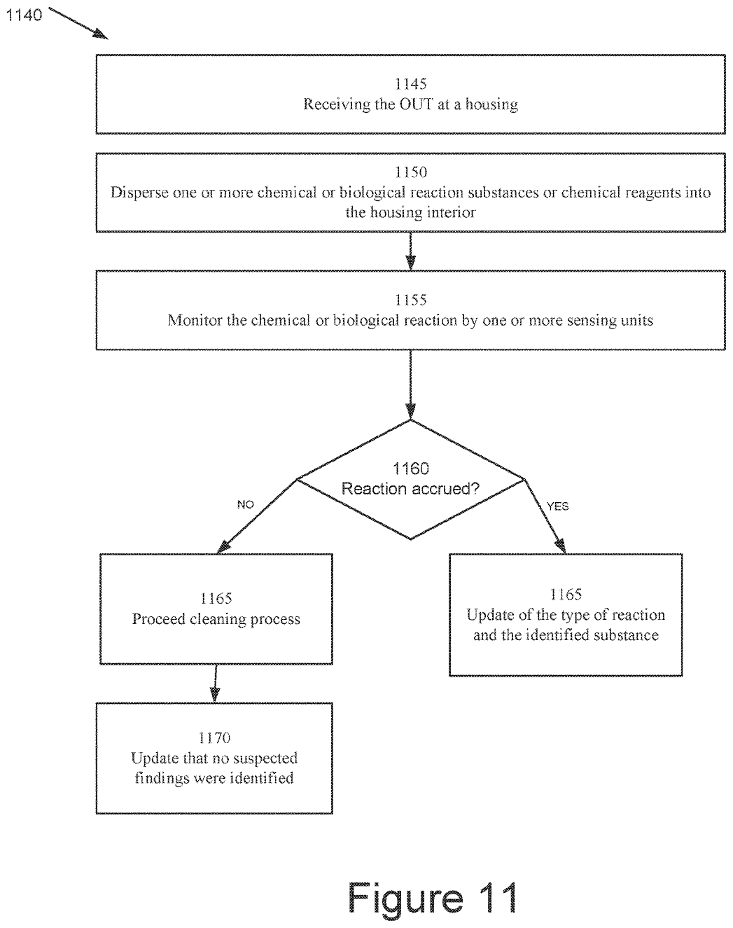

FIG. 11 is a flowchart 1100 illustrating a method for identifying and/or diagnosing one or more substances in a toilet in accordance with embodiments. At step 1105 one or more chemical or biological reaction substances or chemical reagents are dispersed into the interior of a toilet, for example by one or more nozzles such as nozzle of module 360 or the rotating arm. At step 1110 the chemical or biological reaction is monitored by one or more sensing modules such as an imager or any sensor. At the following conditional step 1155 if affirmative, and it is determined by the one or more sensor that a reaction such as a change in color was identified then at step 1120 the device may update via for example the communication module an external device such as a smart phone device of the type of reaction and other details regarding the identified reaction. If a reaction was not identified the cleaning procedure may proceed at step 1125. Alternatively or in combination at step 1130 the device may update that no suspected finding was identified.

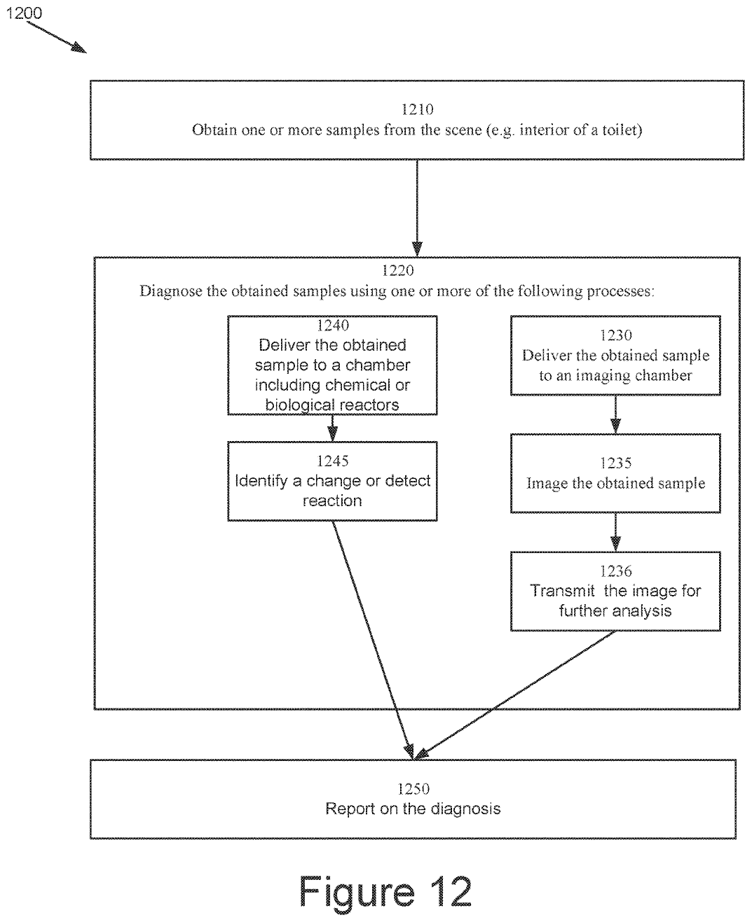

FIG. 12 is a flowchart of a method 1200 for identifying and/or diagnosing one or more substances (e.g. OUT) to characterize the substances found within a scene, in accordance with embodiments. The scene may be one or more of indoor areas such as areas located in factories for example food factories medicine factories, clean rooms of factories, hi-tech industry, heavy industry, light industry, or at outdoor areas such as farms, agriculture such as grain fields open fields such as near or at a sea at a river near or at water reservoir in pipes (e.g. to monitor the water quality), laboratories such as research laboratories, for example food laboratories, medicine laboratories, material laboratories, chemical laboratories, laboratories in industries such as metal industry, for example jewelry industry, printing industries such as 3D printing laboratories, research laboratories such space research laboratories (e.g. NASA laboratories) sea research laboratories particle accelerator laboratory and the like. In further embodiments, the scene may be located in areas where high security level is required to identify explosive and/or hazardous materials such as biological material and to prevent smuggling of drugs, weapons and/or to prevent widespread diseases. Such areas may be for example airports, public or private galleries and salons, offices, hotels, schools army bases hospitals and the like. In accordance with embodiments, the device may be attached to or included in toilets for example in a toilet cleaning device as illustrated herein in FIGS. 1A-1G, 2A-2C, 3A-3I or in other locations in a toilet cell. In further embodiments, the device may be attached to doors such to a door knob. In other embodiments, the device may be included in a security scanning machine such as machine poisoned at the entrance of airports.

The At step 1210 one or more samples of the OUT are obtained, for example the samples may be obtained from an interior of a toilet lavatory seat or a toilet bowel using for example tubule 1010 of a toilet cleaning device as illustrated in FIG. 10A. At step 1220 the obtained samples are diagnosed using one or more of the following processes: at step 1230 the obtained sample is delivered for example via the tubule 1010 or via other pipes to an imaging chamber (e.g. diagnosing module) for imaging the obtained sample. At step 1235 the obtained sample is imaged using for example microscopically and in high resolution to yield one or more images. At step 1236 the images are transmitted for example via the communication module to an external device and/or to a network server processor or to a laboratory for further analyzing the images and/or comparing the images to existing images stored for example at a database. At step 1240 the obtained samples are delivered to a chamber including chemical or biological reactors. At step 1245 if a change or a suspected reaction was identified then at step 1250 the diagnosis is reported including for example the type of diagnose identified, the type of threat etc. For example, the diagnose may include detecting explosive material, drugs or diseases. The finding may be automatically reported to the security authorities.

According to some embodiments, the method 1200 may follow method 1100. For example, method 1200 may be operated if a positive reaction was identified and further analysis is required.

In some cases, the sensors may be placed at the tubule and the sensing procedure may be performed at the tubule.

FIG. 13A shows an isometric view of a device 1300 (e.g. diagnosing module) for characterizing and/or diagnosing one or more substances 1314 in a scene, in accordance with embodiments. The device 1300 may include a housing 1310 (e.g. vessel or chamber or container) having a cavity therein wherein the cavity is configured to contain the substances 1314, an imaging module comprising one or more imagers or an array of imagers such as imagers 1321, 1322 and 1323 for imaging the substances 1314, an illumination module comprising one or more illumination sources such as illumination sources 1325 and an illumination array 1326 for illuminating the substances 1314, a sensor module 1340 comprising one or more sensors of various types for sensing the substances and generating sensory data, a connection interface module 1350 for connecting the device 1300, for example wirelessly, to other devices such a portable device (e.g. communication device) for example through a dedicated connection such a USB connection, wireless connection or any connection known in the art.

According to one embodiment, the housing 1310 may be portable and weight about 200 gr or less gr to 3 kg or more kg and have a volume of about 2×2×2 cm³ to few microns or more so the device may be inserted or attached to small devices such as a toilet cleaning device. For example, the device size may be 1×1×1 cm³ or less. In some cases, the housing 1310 may be carried or lifted by a user and placed in a scene for imaging one or more substances in the scene. In some cases, the housing 1310 may be or may include a sack or a permeable tube filler made of for example fabric or any permeable material known in the art. In an embodiment, the housing 1310 body may be selected from a group consisting of: a pipe, a pipeline, a bottle, a test tube, a permeable tube, a non-permeable tube an open lab tube, a closed lab tube. In an embodiment, the housing 1310 may be in the shape of a pipe, a box or any shape capable of holding the substances 1314. In an embodiment, the housing 1310 may be made of a material for sealing light and/or sound and/or moisture and/or gas or any material for preventing any external material to penetrate into the housing.

According to some embodiments, the housing 1310 may be made of one or more of the following materials: glass; aluminium; metal; steel; steel alloy; gold; silver; polymers; plastic; paper; nylon; rubber; wood, and the like.

In some embodiments, the housing bottom surface 1318 may include a dedicated holder 1319 such as a vial for holding the sample (e.g. the substances 1314). In some embodiments, the housing bottom surface 1318 may hold the sample. In some embodiments, the bottom surface 1318 may be or may include a surface for holding the substances. In some cases, the holder 1319 may be square or rectangle shaped or in any other shape.

In some embodiments, the housing 1310 may include one or more opening to insert or collect the substances. For example, the housing 1310 may include an opening window 1317 at the upper surface of the housing 1310 which may be for example opened automatically to collect the substances.

The sensor module 1340 may include one or more sensors of various types attached for example to the housing 1310 inner surface, in accordance with embodiments. The sensor module 1340 may include one or more of a pressure sensor, humidity sensor, acidity sensor, proximity sensor, size gauge sensor, material meter sensor, explosive detection sensor, virus detection sensor, motion sensor, light intensity sensor, spectrometer, or any type of sensor known in the art. In an embodiment, the device 1300 may include two sensors such as sensors 1341 and 1342 attached to the inner side of the housing 1310 in proximity or above the substances 1314 configured to sense the substances and provide sensory data including for example spectral data, temperature of the substances and/or temperature inside the device 1310 etc. In some cases, the sensors may position in proximity to the substances 1314 and some may be attached to the upper surface of the housing. For example, one or more biological sensors for sensing tissues, microorganism, organelle, receptor, enzyme, antibody, nucleic acids or electromagnetic addition may be positioned in proximity to the substances (e.g. few mm) to correctly sense the substances 1314.

Other examples of sensors which may be used include for example: acceleration sensor; conductivity sensor; CO2 sensor; current sensor; force sensor, humidity sensor; light sensor; microphone; sound sensor; pressure sensor; sheave sensor; temperature sensor such as surface temperature sensor; UVA or UVB sensor; distance sensor; spirometer; oxygen sensor; voltage sensor; drop counter sensor; EKG sensor; CO2 sensor; magnetic field sensor; melted oxygen sensor; IR sensor; heartbeat sensor; color sensor; gyroscope; soil moisture sensor; ultrasonic sensor; proximity senor; alcohol sensor; touch sensor and the like. For example, sensory data captured by these sensors are transmitted to the processor to identify and characterize the OUT, for example by comparing the obtained sensory data to existing sensory data.

Specifically, one or more sensors such as the alcohol sensor may monitor one or more objects inserted into the device or the number of drops added to the OUT 1314 and report on any change, for example in real time identified in the device and at the object (e.g. sensory data). The sensory data and the images are analyzed by the processors and the resulted identifications are transmitted to an external device such as the mobile communication device.

In an embodiment, the imaging module includes one or more imagers configured to capture images or videos of the substances 1314 in high resolution from different angels and distances. In some cases, each of the imagers may include a high-resolution camera of a resolution and focus of between 1000 to 10 million pixels and more. In some cases, the imagers may have a magnification which is configured to identify the texture and/or shape of the substances such as the texture and/or shape of a single or plurality of biological cells or the shape of a bacterium; virus; fungus; organic material, chemical material; radiologic material or the like.

In some embodiments, the imagers are configured to image the substances using super-resolution microscopy systems and methods such as stimulated emission depletion microscopy systems and methods and/or single-molecule microscopy single-molecule microscopy. Such methods may be found in U.S. Pat. No. 9,772,285 and US20100176307 which is incorporated herein by reference. In some cases, the imagers are configured to image the substances in sub illumination in ultra violet (UV by) video (e.g. to capture a movement of the OUT) or using radio frequency (RF) imagers including RF transmitters and receivers to obtain RF reflections from the substances or using fluorescent illuminators. In some embodiments, the imagers are configured to image the substances using optical tweezers (e.g. single-beam gradient force trap). Optical tweezers are scientific instruments that use a highly focused laser beam to provide an attractive or repulsive force (typically on the order of piconewtons), depending on the relative refractive index between particle and the surrounding medium, to physically hold and move microscopic objects similar to tweezers. They are able to trap and manipulate small particles, typically order of a micron in size, including dielectric and absorbing particles. In accordance with embodiments, the optical tweezers are configured to hold and move the substances to image and diagnose the substances until the substances are completely or partially identified. In some embodiments, the imaging module may include one or more Internet of Things (IoT) cameras and sensors configured to identify in the substances a single cell in a single photograph using: local analysis/cloud analysis/Internet of things/neural network/artificial intelligence.

In some embodiments, the imaging module may use filtering methods to characterize the OUT the method may include technics such as “Bacterial capture by resin-linked oligy-acyl-lysines” methods as explained in http://t3.trdf.co.il/show_invention/44 and incorporated herein by reference.

According to one embodiment, the imaging module may include three imagers such as imagers 1321, 1322 and 1323 attached to the inner sides of the housing 1310 and above the substances (e.g. two imagers 1321 and 1322 at opposite sides and another imager at the upper section of the housing).

In accordance with embodiments, the imagers may be or may include one or more of the following cameras 2D or 3D cameras: 46-PS2-LIB Leica; Leica S (Typ 007); Mamiya Leaf Credo 80 Digital backSeitz 6×17 Panoramic Camera; Hasselblad H5D-400c; H-series H4D-60 Hasselblad; PhaseOne Phase one 645 DF; Genie Nano-CXP; 3D camera such Z-Trak 3D Laser Profiler or other cameras such as: Genie Nano-CXP M4090, Genie Nano-CXP M4090-NIR, Genie Nano-CXP M5100, Genie Nano-CXP M5100-NIR, Sony a7r alpha, sony a7 series, Nikon D5, Nikon D850, Nikon z7, Nikon z8, Nikon z9y, nikon z10, Nikon z1, Canon EOS-1D X Mark II, Canon EOS 80D, Canon Rebel SL2/EOS 200D, Canon EOS M50, Canon EOS R, Canon EOS 5D Mark IV and the like.

In some embodiments, one of the imagers such as imager 1323 may be attached to the housing upper section (e.g. roof) and the other imagers (e.g. imagers 1321 and 1322) may be attached to the housing sides for imaging the substance in an angle of about 45°. In some cases, imager 1323 may be attached to a track to enable the imager to slide up and down in respect to the substance 1314. In some cases, the imaging module may be in communication with one or more processors for controlling the movement of the imagers and activating the imagers (e.g. when and from which angle to image the substance). In some cases, the imagers may be controlled autonomously using Deep Neural Network (DNN) or Artificial Intelligence (AI).

FIG. 13B shows an isometric view of a device 1301 for characterizing and/or diagnosing one or more substances in a scene, in accordance with another embodiment. Device 1301 present all or some of the elements of aforementioned device 1300 but further includes: a gas supply device 1308 for storing and inserting gas such as oxygen and/or nitrogen into device 1300; power supply module 1315 such as energy saving power supply, a mechanical module 1309 configured to insert/take out objects such as substances or any type of materials into device 1301 via one or more openings such as opening window 1317 at the upper surface of the housing 1310; a thermometer 1320 for measuring the temperature inside the device 1301 and a an air pump opening 1324 to be connected to an air pump for creating vacuum in the device 1301. In an embodiment, the gas supply device 1308 may include a gas tank and a pump connected to the housing via one or more openings such as opening 1324 to suck air from the housing and create a vacuum inside the housing or to insert gas from the gas tank into the housing.

In an embodiment, the gas supply device 1308 is configured to insert one or more of the following gas: oxygen, nitrogen, CO2. In some cases, the device 1308 may include an electronic tap and regulators for controlling the amount of gas inserted into the housing (e.g. and which effects the substances 1314). For example, to detect listeria, the processor may instruct device 1308 to insert oxygen into the housing and the one or more processors and imagers may monitor the effect on the substances.

In an embodiment, the mechanical module 1309 may include a mechanical arm 1311 and/or a syringe 1312 for inserting one or more materials into the housing for example via opening 1317.

In an embodiment, the power supply module 1315 may include a power source. In some embodiments, the device 1301 is powered by a power supply from an external source. In some embodiments, the device 1301 has an independent power supply. In some embodiments, the device also includes a power source (e.g. a battery or power supply). In some embodiments, the device is powered by a power supply from a consumer's hand held device (e.g. a cell phone). In some embodiments, the power source may be an energy saving power source including for example solar panels 1327.

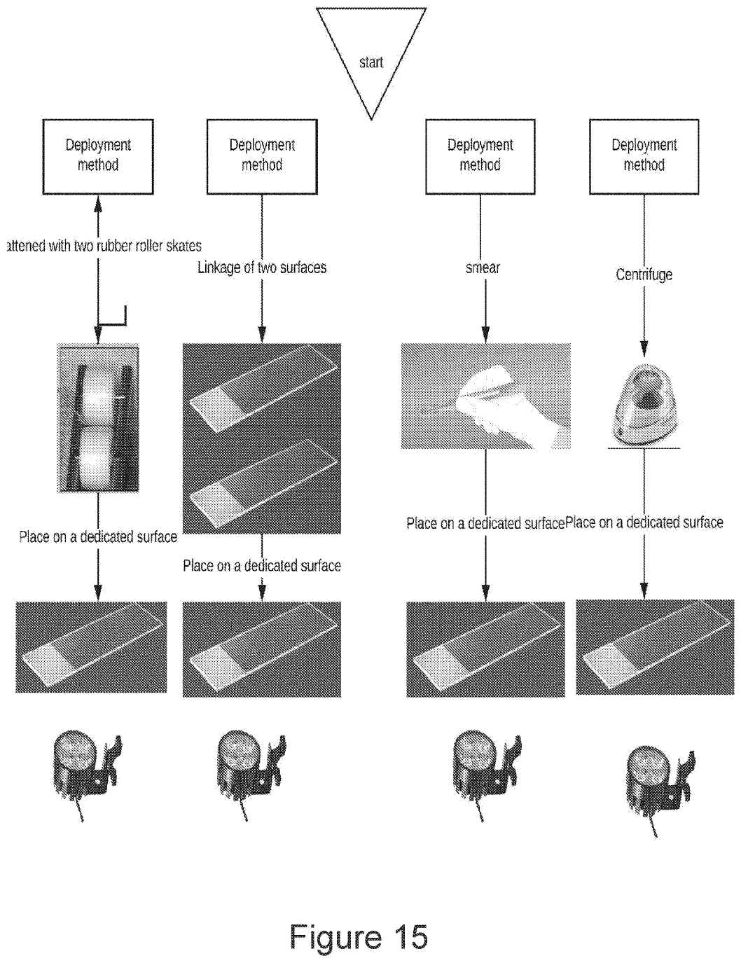

FIG. 13C shows an isometric view of a device 1302 for characterizing and/or diagnosing one or more substances in a scene, in accordance with another embodiment. Device 1303 present all or some elements of the aforementioned devices 1301 and 1302 but further includes an insertion module 1313 configured to receive and insert the substances into the device 1302. In an embodiment, the insertion module 1313 may include two cylinder shaped modules 1331 and a notch 1339 formed between them. In some cases, the substances 1314 may be sandwiched and/or enveloped in a holding element 1337 and the two cylinder shaped modules 1331 may be rotated to receive the substances 1314 and autonomously insert the substances into the device 1302. In some cases, once the substances are in the device 1302 they may swiftly fall into the holder 1319. Visual examples of insertion modules which may be attached or included in device 1302 or other devices such as devices 1300-1306 are illustrated in FIG. 15.

FIG. 13D shows an isometric view of device 1303 for characterizing or diagnosing one or more substances in a scene, in accordance with another embodiment. Device 1303 present all or some of the elements of aforementioned devices 1300, 1301 and 1302 but further includes: a mechanical arm for placing the OUT in a holder (e.g. glass/polymer or other material) at a required position for further imaging the OUT by the imagers from one or more angles, a machine which separates substances of different densities, for example a centrifuge 1333 and a motor 1334 for activating the centrifuge and the mechanical arm. For example, the OUT 1314 included in the holding element 1337 and may be inserted to the housing and further to the centrifuge for preparing and mixing the OUT, in accordance with embodiments.

FIG. 13E shows a diagram of a system 1305 such as a network system for detecting and/or diagnosing and/or alerting on one or more substances (e.g. Object under test (OUT)) such as chemical-biological-explosive (CBE) or other substances which may be or may include for example threats on-site and allow for immediate responders to mitigate spread, risk, and loss, in accordance with embodiments. System 1305 comprises a device 1306 which may be in wireless communication 1316 with a cloud based remote server system 1361 and additional sub-systems which may be external to the device 1306. System 1305 presents all elements of the aforementioned system 700 but further includes additional elements, modules, sub-modules and sub-systems, in accordance with embodiments. In some cases, device 1306 may also be in wireless communication with a hand held device such as a mobile communication device for transmitting alerts to security services. In some cases, the hand held device may include data storage subsystems and/or data processing subsystems.

Device 1306 present all or some of the elements of aforementioned devices 1300, 1301, 1302 and 1303 but further includes: one or more cameras such as cameras 1321, 1322, 1323 and video camera 1360 for capturing video images of the OUT, one or more projectors such as projector 1362 for illuminating the scene (e.g. and the substances 1314 in the device), one or more heating elements such as heater 1364 configured to heat the scene (e.g. the interior of housing 1310), a cooling device 1366 configured to cool the scene (e.g. the interior of housing 1310), an air system 1368 comprising an air pump 1370 configured to suck air from the housing interior and create vacuum inside the housing to take out the substances 1314 from the housing 1310 and/or a pumping device 1372 such as a vertical pump for releasing out the substances from the device 1306, a fan 1374 such as a triple fan blades for cooling the housing, a fluid nitrogen, one or more opening 1377 for inserting working tools such as working gloves 1378 configured to sterilely move or take the substances or control other modules in the device 1306 by a user, one or more fluid injectors 1380 a patch manager 1382, an agitator 1384, and a database such as internal database 1383 for storing captured images and sensory data and/or existing data including existing images to be compared to the captured images to identify and characterize the OUT.

In some cases, the pumping device 1372 may be controlled by the processor when and the number of substances which should be released from the housing.

In some cases , the fluid injectors 1380 may be controlled by the processor to insert one or more fluids to the housing such as sweet fluids (e.g. sugar).

In some cases, the device 1306 may include a scanner 1343 for scanning the OUT 1314. The scanner may be controlled by the processor and may scan the OUT following the imaging of the OUT.

In some cases, the imager and camera such video camera 1360 may be connected to a mechanical arm which may move the video up and down, for example automatically and/or autonomously, closer to the OUT 1314 to capture images of the OUT from short distance.

In some embodiments, the device 1306 may include or may be in communication, for example in wireless communication, with one or more external modules such as control module 1359 for controlling and processing data such as images and sensory data received from the device 1306. The control module 1359 may include: a communication module 1388 comprising: a server 1361, communication circuity 1389 for connecting the device 1306 to external devices; a display module 1363 comprising: an audio device 1391 and/or a display 1392 for visually displaying and/or audio playing the characterizing results of the OUT; a printer 1397; a processing module 1365 comprising: database 1387 and one or more processors such as CPU 1399 or GPU for receiving and processing data comprising, for example, one or more images captured by the imagers of the device 1306 and/or sensory data obtained by one or more sensors of the device 1306. In some cases, the processing module 1365 may include: a power source 1381 one or more IoT cameras 1349 and an IoT medical emergency module 1396. In some cases, these modules may be included in a mobile device such as a telephone mobile device or at a server such as a server 1361.

In some embodiments, the communication module 1388 and/or control module 1359 may be the communication module as illustrated in FIG. 7A which is configured to transmit the collected data to a cloud-based server which is configured to analyze the transmitted data and/or to transmit the collected data and/or the analyzed data to other remote systems and modules. For example, the collected data may be transmitted via VPN or via virtual private cloud (VPC) server or via satellite connection 1394 to medical and/or emergency modules such as IoT medical emergency module 1396.

In accordance with embodiments, the one or more processors such as the CPU 1399 or GPU (Graphics Processing Unit) are configured to identify a single cell in a single photograph using one or more of: local analysis, cloud analysis, Internet of things, neural network such as a Deep Neural Network (DNN), Artificial Neural Networks (ANN) and artificial intelligence.

In accordance with embodiments, the ANN is configured to “learn” to perform tasks by considering examples, generally without being programmed with any task-specific rules. In an embodiment, the ANN may identify the substances without prior knowledge. Instead, they automatically generate identifying characteristics from the learning material that they process. An ANN is based on a collection of connected modules or nodes called artificial neurons, which loosely model the neurons in a biological brain. Each connection, like the synapses in a biological brain, can transmit a signal from one artificial neuron to another. An artificial neuron that receives a signal can process it and then signal additional artificial neurons connected to it. In common ANN implementations, the signal at a connection between artificial neurons is a real number, and the output of each artificial neuron is computed by some non-linear function of the sum of its inputs. The connections between artificial neurons are called ‘edges’. Artificial neurons and edges typically have a weight that adjusts as learning proceeds. The weight increases or decreases the strength of the signal at a connection. Artificial neurons may have a threshold such that the signal is only sent if the aggregate signal crosses that threshold. Typically, artificial neurons are aggregated into layers. Different layers may perform different kinds of transformations on their inputs. Signals travel from the first layer (the input layer), to the last layer (the output layer), possibly after traversing the layers multiple times. The original goal of the ANN approach was to solve problems in the same way that a human brain would. However, over time, attention moved to performing specific tasks, leading to deviations from biology. Artificial neural networks have been used on a variety of tasks, including computer vision, speech recognition, machine translation, social network filtering, playing board and video games and medical diagnosis.

In some cases, the device 1306 may include one or more magnets for moving the substances 1314 in accordance with instructions received from the processor or manually by a user.

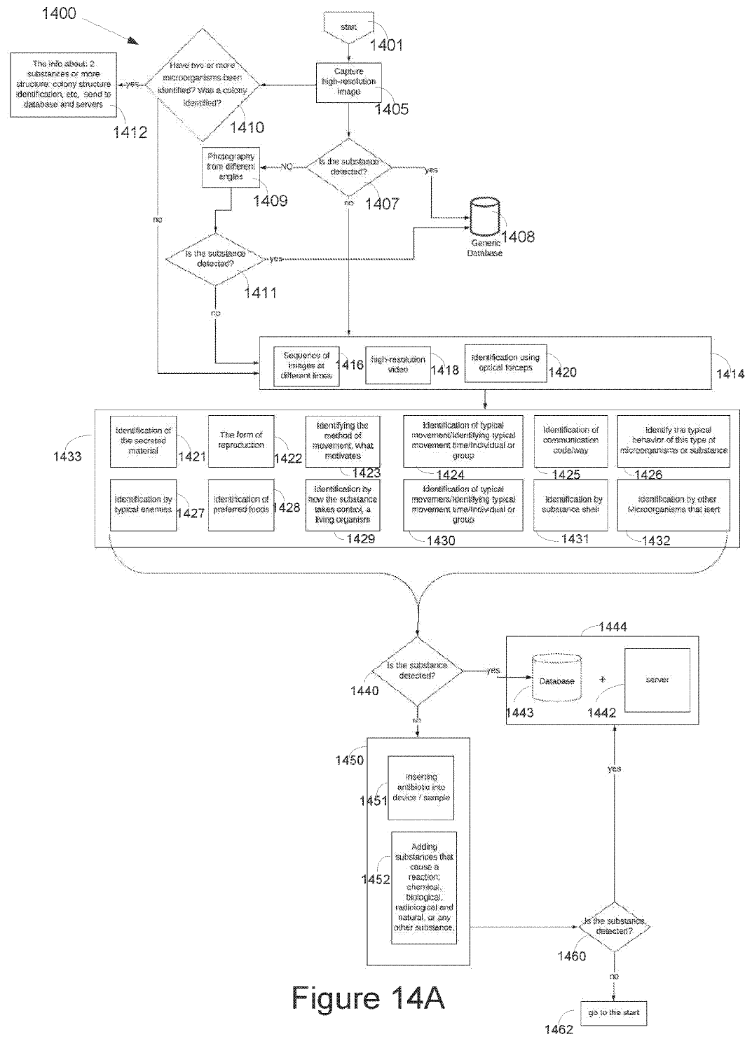

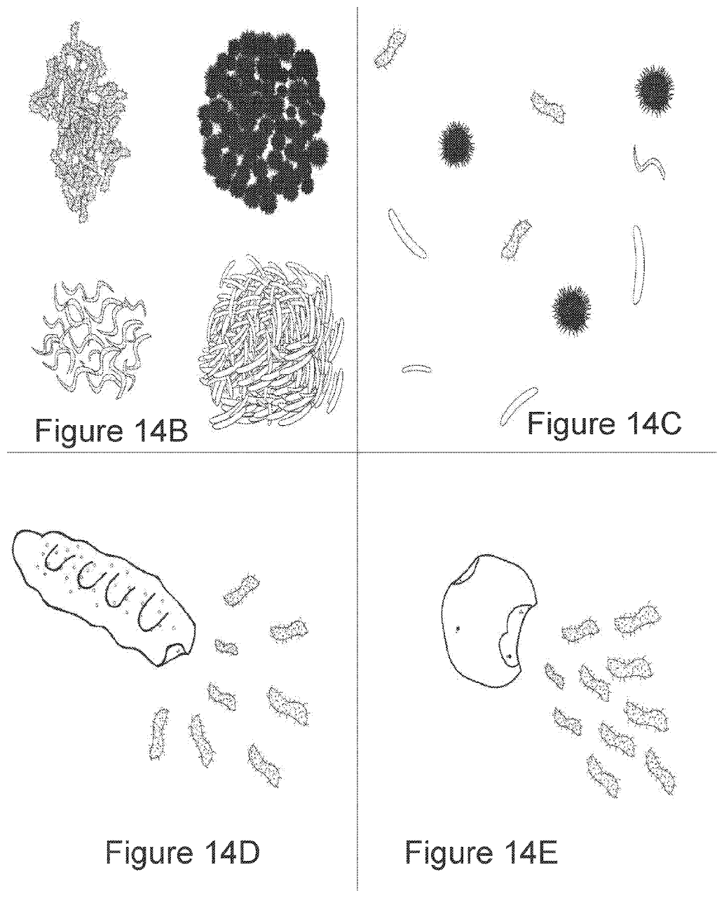

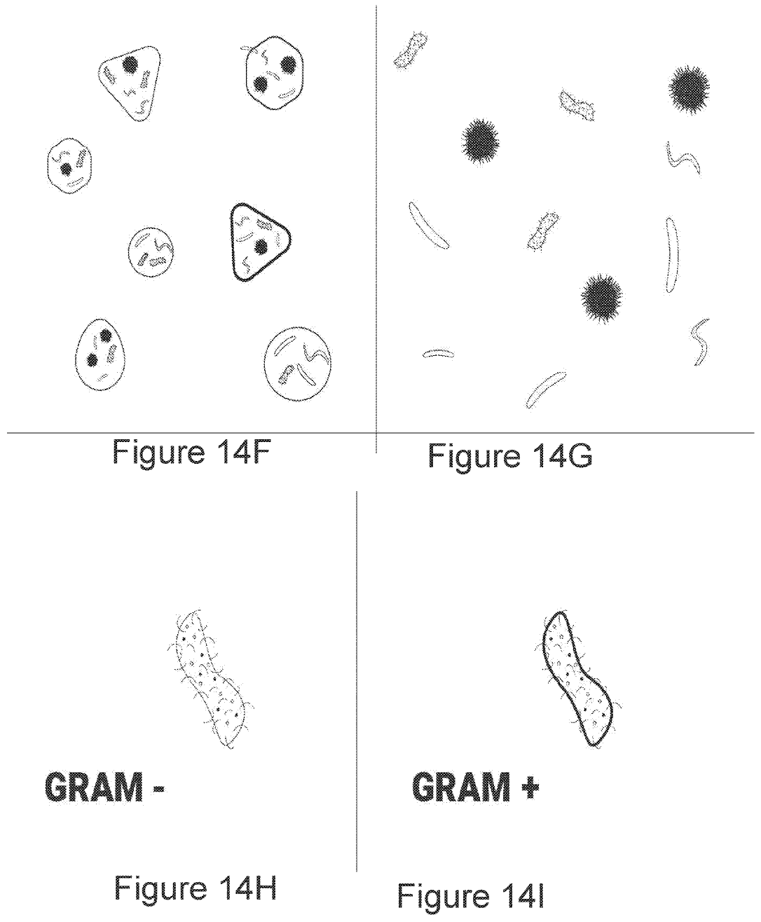

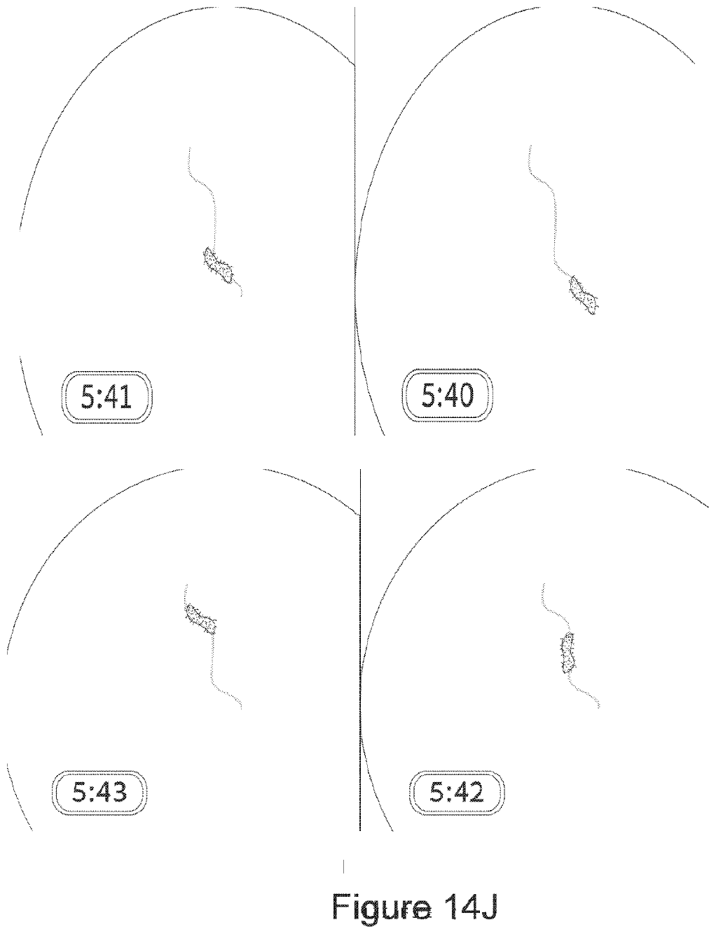

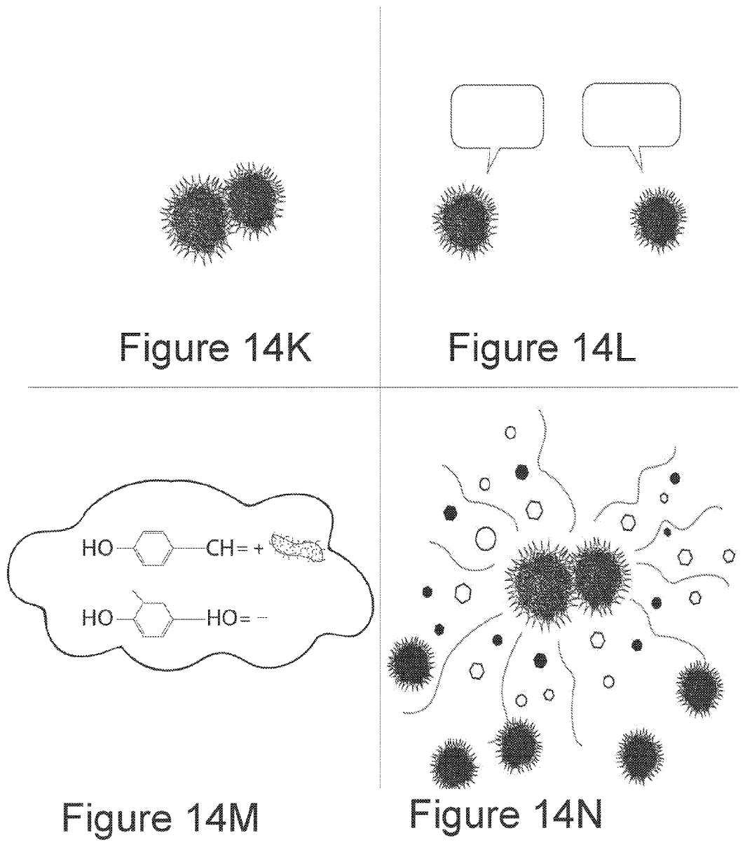

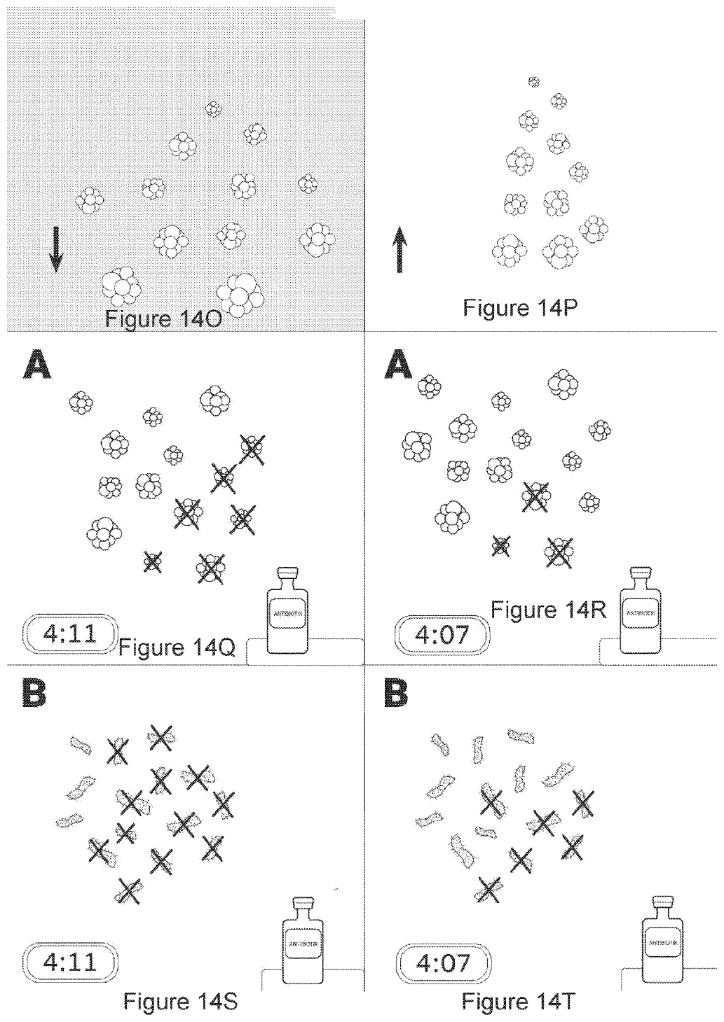

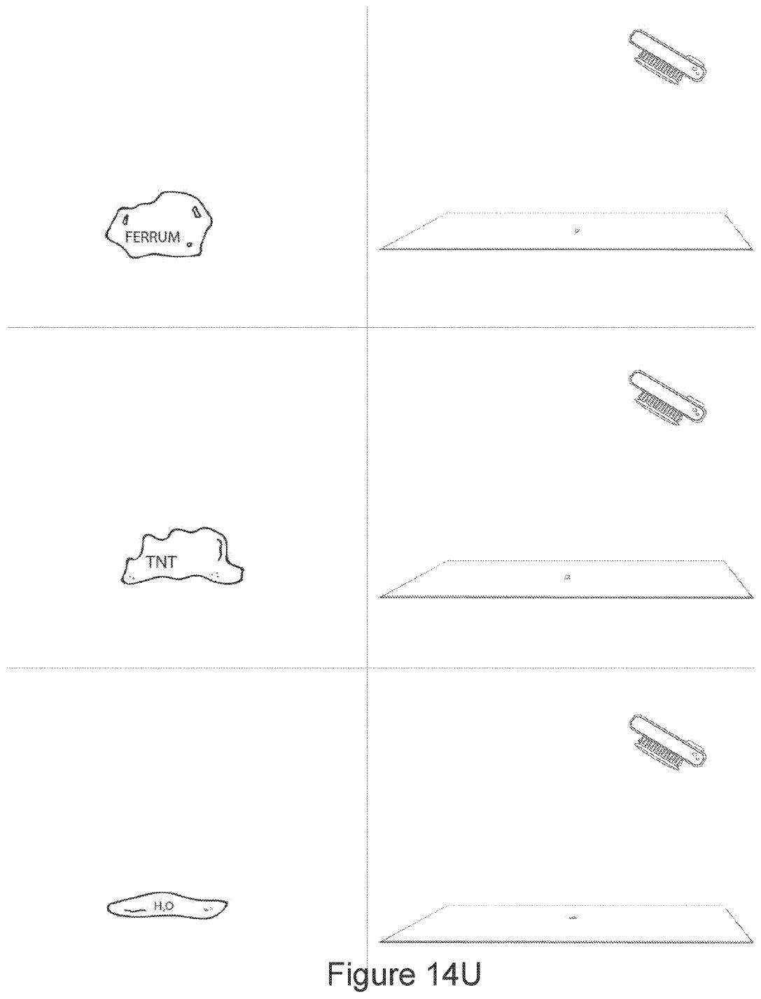

FIG. 14A shows a flowchart of a method 1400 for characterizing and/or diagnosing one or more substances or objects in a scene using one or more sensors and imagers having high resolution capabilities, in accordance with embodiments. The images may be captured by one or more imagers such as the imagers illustrated in FIGS. 13A-13E.

At step 1401 the imaging process starts and the system's modules are activated. In some cases, the system's modules such as the imagers and sensors are autonomously and/or automatically activated for example once the substances are detected and/or once a movement in the scene or in the device is identified. At step 1405 one or more high resolution images of the substances are captured, for example by the imagers illustrated in FIGS. 13A-13E.

At step 1410 the captured images are analyzed, for example by the system's one or more processors, to identify the substances and a binary decision is made regarding the identification result. If the affirmative and the identification result of the substances includes two or more identified microorganism and/or or a colony of cells then at step 1412 the identification result including for example the structure (e.g. colony structure) of the substances and other details such as size, amount and type of identified cells (e.g. type of the disease and identified microbe) are transmitted to a local or external database (e.g. via one or more servers) for further analysis or for updating the relevant modules on the identification results. If negative and not more than a single microorganism and/or a colony was not identified then at step 1414 additional identification actions are operated, for example by a device such one or more of devices 1300-1306, to identify the substances. In accordance with embodiments, the additional identification actions 1414 may include one or more of: capturing a sequence of images at different time intervals 1416 to yield subsequent images which allow tracking and analyzing changes in substances structure in real time as illustrated in FIGS. 14B-14E; capturing high-resolution video images 1418 ; using optical forceps 1420 to move the substances while imaging the substances till they are identified. Specifically, the step of using optical forceps includes (1420), in accordance with embodiments, using optical trapping of dielectric particles by a single-beam gradient force trap. The process may include using negative light pressure. Trapping may be observed over the entire range of particle size from 10 um to −25 nm in water. Use of the new trap extends the size range of macroscopic particles accessible to optical trapping and manipulation well into the Rayleigh size regime. Application of this trapping principle to atom trapping is considered.

In accordance with embodiments, the step of the additional identification actions (1414) may include one or more of the following diagnostic actions and analysis methods which may be preformed on the substances and the obtained video images or sequence of images: identifying the movement of the cell and/or what motivates the movement (1421); the form of reproduction (1422); Identifying the movement of the OUT what motivates (1423); identification of typical movement/identifying typical movement time/Individual or group (1424); Identification of communication code/way (1425); identify the typical behaviour of this type of microorganisms or substance (1426); Identification by typical enemies (1427); Identification of preferred foods (1428); Identification by how the substance takes control, a living organism (1429); Identification of typical movement/Identifying typical movement time/Individual or group (1430); Identification by substance shell (1431); Identification by other microorganism which is inserted to the OUT (1432); Identification by light or dark preference (1433). Examples of additional identification actions (1414) are illustrated in FIGS. 14B-14N. At the following step 1440 a binary decision is made regarding the identification result of the substances according to the analysis of the captured images/video based on one or more of the diagnostic methods 1421-1433. If the substances are identified then at step 1444 the identification result is transmitted to a local or remote database 1443, for example via server 1444. If the substances are not identified then at step 1450 one or more of the following additional diagnostic methods may be performed: inserting antibiotic into the device and/or to the substances (1451) as illustrated at FIG. 14O-14T; adding one or more additional substances that cause a reaction, the additional substances may be one or more of chemical, biological, radiological and natural, or any other type of substance (1452).

At the following step 1460 a binary decision is made regarding the identification result of the substances according to the analysis of the captured images/video based on one or more of the diagnostic methods 1451-1452. If the affirmative and the substances are identified then at step 1444 the identification result is transmitted to a local or remote database 1443, for example via server 1444. If the substances are not identified then at step 1462 the diagnostic method is repeated and proceeds to the starting step 1401 till the OUT is diagnosed, in accordance with embodiments.

Alternatively or in combination, following the image capturing step 1405 a binary decision 1407 is made regarding the identification result. If the affirmative and the substances were identified then at step 1408 the identification result including for example the structure (e.g. colony structure) of the substances and other details such as size, amount and type of identified cells (e.g. type of the disease and identified microbe) are transmitted to a generic database 1408 such as local or external database (e.g. via one or more servers) for further analysis or for updating the relevant modules on the identification results. If negative and the substances are not identified then at step 1409 the substances are images from multiple angles (e.g. using for example imagers 1321-1323 of FIGS. 13A-13E) and a binary decision is made regarding the identification result (1411). If the affirmative and the substances are identified by analyzing the multiple angle images then the identification result including for example the structure (e.g. colony structure) of the substances and other details such as size, amount and type of identified cells (e.g. type of the disease and identified microbe) are transmitted to a generic database 1408 such as local or external database (e.g. via one or more servers) for further analysis or for updating the relevant modules on the identification results. If the substances are still not identified then the at step 1414 additional identification actions are operated, for example by a device such one or more of devices 1300-1306, to identify the substances.

In accordance with embodiments, the high-resolution captured images of the substances may be analyzed using one or more processors to identify the substances. For example, the identification of a single cell of the imaged substances may be based on the structure and/or the cell's material. In other words, the cell is identified by its characteristic structure, e.g. if it is a bacterium it may be identified according to the cell's scallions; cell shape; cell size; cell thickness etc. In cases where the cell is a material cell, then it may be identified according to the classical structure of the cell and a possible structure of the cell depending on the size of the cell's particle.

FIGS. 14B-14T illustrates one or more visual examples of the diagnostic methods used while analysing the captured images and/or video images of the substances as mentioned herein in respect to flow chart steps illustrated in FIG. 14A, in accordance with embodiments. The visual images illustrated in FIGS. 14B-14T are an optical magnification of the captured image and/or images captured using high resolution imagers (e.g. 10000 pixels or more) for example in several orders of magnitude. In some cases, an identification of a single cell of an OUT or one or more substances may be based on the cell structure and/or the cell's material. In other words, the cell is identified by its characteristic structure: if it is a bacterium: according to the scallions, by cell shape, by cell size, by cell thickness. If it is a material cell, then according to a classical structure and a possible structure, depending on the size of the particle.

FIG. 14B shows an example of visual images of the substances wherein the substances are identified according to the shape of the colony. For example, in FIG. 14B four types of different colonies are identified while FIG. 4C shows an optical magnified image of the substances where the substances may be identified according to the cell shape of each substance. FIG. 14D shows a captured image of one or more substances wherein the substance is food and the food is identified according to one or more of: the type of the food; the food's bacterium; the size and direction of the food and bacterium. Specifically, the substance may be identified by one or more microorganisms of the substance or microorganisms related to the substance. For example, repellent or other threatening materials. In other embodiments, the substance may be identified according to the movement of the cells of the substance (e.g. direction of the movement; its characteristic shape; its distance at a given time). In further embodiments, the substance may be identified by potential enemies and flight of microorganisms from them.

FIG. 14E shows an example of captured images of a substance wherein the substance is a fruit (e.g. apple) and the substance's related bacterium are identified according to the type of food and the colony. For example, the substance may be identified based on the microorganisms by preferred food or preferred space or any other preferred material and/or according to the movement of the cells to the substance a processor can identify the direction of the movement, its characteristic shape, its distance at a given time, and so on.

FIGS. 14F and 14G show an example of an identification of the OUT based on analysing the captured images and detect where one or more microorganisms of the OUT may develop and/or and/or future evolution and/or by comparison to other well defined microorganisms with the same or nearly identical cell structure or the same family or the same type of the imaged microorganisms elsewhere in the world evolved into the form shown in the FIG. 14F.

Specifically, FIG. 14G shows an example of a captured magnitude image of one or more cells of a substance before the cells are interconnected to one another, in accordance with embodiments. The substance may be identified based on each or some of the substances' microorganism structure and characteristics, e.g. switch, worm, woollen, pellets, and so on. FIG. 14F shows an example of a captured magnitude image one or more cells of a substance following the cells formed a colony and the way and shape the microorganism are interconnected to one another. For example, the substance may be identified according to the structure of the colony and its characteristic shape. In further embodiments, the substance of FIG. 14F may be identified by monitoring the substances and related microorganism over time (e.g. using for example imagers 1321-1323) and the way they interconnect with one another. For example, the switch-like shape bacteria would prefer to be near the spherical shape bacteria that would prefer to interconnect to the worm-like shape bacteria. This will create a typical bacterial bacterium. Accordingly, the substance may be identified according to the way the microorganism interconnect to one other and/or according to the resulted colony and/or according to their movement in their preferred direction.

FIGS. 14H and 14I show a magnified image of a substance, wherein the substance is identified by measuring the substances cells thickness and if the cell's Gram-positive or Gram-negative, in accordance with embodiments.

FIG. 14J shows a sequence of images captured over time of an OUT, in accordance with embodiments. The OUT may be identified according to the movement of one or more cells/microbe/chemical or any portion of the OUT over time. For example, in some cases, the movement duration and/or direction and/or speed and/or distance may be monitored and measured over time to identify the OUT.

FIGS. 14K and 14L show an example of identification of an OUT based on microbiology identification e.g., the measured distance between cells may be used to identify the OUT. In some cases, one or more microorganisms may communicate with other microorganisms, this communication may be tracked by the sensors and imagers in accordance with embodiments and may be further analyzed to identify the OUT.

FIG. 14M shows an exemplary analysis result of a captured image of the OUT wherein the analysis is based on identifying one or more molecules found in the cells of the OUT such as DNA or RNA, in accordance with embodiments. Specifically, the cell may emit one or more cells and these cells may be used to identify the OUT. In some cases, the images may be analyzed to identify and detect the next action of the cell and microorganism.

FIG. 14N shows an example of communication between imaged microorganisms. To communicate with one another the microorganisms use chemicals, chemical clues, organelles, chemical or biological probes, energy, and other communication ways which may be monitored and identified by the imagers, sensors, and other modules to identify the OUT, in accordance with embodiments.

FIG. 14O shows an example of an identification of the OUT based on analysing the captured images and detection of microorganism movement (e.g. direction and/or speed) of the OUT with no illumination (e.g. in darkness) while FIG. 14P shows the movement while illuminating the OUT (e.g. in light). In accordance with embodiments, the images of the OUT with/without illumination are compared to one another to identify the OUT. The identification, in some cases, may include monitoring the OUT from various angles to identify the microorganism movement (e.g. direction and/or speed).

According to some embodiments, one or more materials may be added to the substances and the processor may comprise instructions to track changes in the captured images and/or sensory data as a result of the insertion of said materials. FIGS. 14Q-14T show an example of an identification of the OUT based on analysing the captured images and monitoring the number of microorganisms which died and when, while adding one or more materials (e.g. antibiotic, oxygen and/or nitrogen) to the OUT or to the OUT surrounding, in accordance with embodiments.

FIG. 14U shows another example of magnified high-resolution images of different OUT (e.g. TNT, water) captured by the device. The captured images may be analyzed by one or processors which compare the images to data stored a database which includes lookup tables or predefined data of known substances such as features of millions of substances (e.g. size, color, movement etc.).

In further embodiments, the processing module may be a digital processing device including one or more hardware central processing modules (CPU) that carry out the device's functions. In still further embodiments, the digital processing device further comprises an operating system configured to perform executable instructions. In some embodiments, the digital processing device is optionally connected to a computer network. In further embodiments, the digital processing device is optionally connected to the Internet such that it accesses the World Wide Web. In still further embodiments, the digital processing device is optionally connected to a cloud computing infrastructure. In other embodiments, the digital processing device is optionally connected to an intranet. In other embodiments, the digital processing device is optionally connected to a data storage device.

In accordance with the description herein, suitable digital processing devices include, by way of non-limiting examples, server computers, desktop computers, laptop computers, notebook computers, sub-notebook computers, netbook computers, netpad computers, set-top computers, handheld computers, Internet appliances, mobile smartphones, tablet computers, personal digital assistants, video game consoles, and vehicles. Those of skill in the art will recognize that many smartphones are suitable for use in the system described herein. Those of skill in the art will also recognize that select televisions with optional computer network connectivity are suitable for use in the system described herein. Suitable tablet computers include those with booklet, slate, and convertible configurations, known to those of skill in the art.

In some embodiments, the digital processing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing. Those of skill in the art will also recognize that suitable mobile smart phone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®.

In some embodiments, the device includes a storage and/or memory device. The storage and/or memory device is one or more physical devices used to store data or programs on a temporary or permanent basis. In some embodiments, the device is volatile memory and requires power to maintain stored information. In some embodiments, the device is non-volatile memory and retains stored information when the digital processing device is not powered. In further embodiments, the non-volatile memory comprises flash memory. In some embodiments, the non-volatile memory comprises dynamic random-access memory (DRAM). In some embodiments, the non-volatile memory comprises ferroelectric random access memory (FRAM). In some embodiments, the non-volatile memory comprises phase-change random access memory (PRAM). In other embodiments, the device is a storage device including, by way of non-limiting examples, CD-ROMs, DVDs, flash memory devices, magnetic disk drives, magnetic tapes drives, optical disk drives, and cloud computing based storage. In further embodiments, the storage and/or memory device is a combination of devices such as those disclosed herein.

In some embodiments, the digital processing device includes a display to send visual information to a user. In some embodiments, the display is a cathode ray tube (CRT). In some embodiments, the display is a liquid crystal display (LCD). In further embodiments, the display is a thin film transistor liquid crystal display (TFT-LCD). In some embodiments, the display is an organic light emitting diode (OLED) display. In various further embodiments, on OLED display is a passive-matrix OLED (PMOLED) or active-matrix OLED (AMOLED) display. In some embodiments, the display is a plasma display. In other embodiments, the display is a video projector. In still further embodiments, the display is a combination of devices such as those disclosed herein.

In some embodiments, the digital processing device includes an input device to receive information from a user. In some embodiments, the input device is a keyboard. In some embodiments, the input device is a pointing device including, by way of non-limiting examples, a mouse, trackball, track pad, joystick, game controller, or stylus. In some embodiments, the input device is a touch screen or a multi-touch screen. In other embodiments, the input device is a microphone to capture voice or other sound input. In other embodiments, the input device is a video camera to capture motion or visual input. In still further embodiments, the input device is a combination of devices such as those disclosed herein.

In some embodiments, the system disclosed herein includes one or more non-transitory computer readable storage media encoded with a program including instructions executable by the operating system of an optionally networked digital processing device. In further embodiments, a computer readable storage medium is a tangible component of a digital processing device. In still further embodiments, a computer readable storage medium is optionally removable from a digital processing device.

In some embodiments, a computer readable storage medium includes, by way of non-limiting examples, CD-ROMs, DVDs, flash memory devices, solid state memory, magnetic disk drives, magnetic tape drives, optical disk drives, cloud computing systems and services, and the like. In some cases, the program and instructions are permanently, substantially permanently, semi-permanently, or non-transitorily encoded on the media. In some embodiments, the system disclosed herein includes at least one computer program, or use of the same. A computer program includes a sequence of instructions, executable in the digital processing device's CPU, written to perform a specified task. Computer readable instructions may be implemented as program modules, such as functions, objects, Application Programming Interfaces (APIs), data structures, and the like, that perform particular tasks or implement particular abstract data types. In light of the disclosure provided herein, those of skill in the art will recognize that a computer program may be written in various versions of various languages.

The functionality of the computer readable instructions may be combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof. In some embodiments, a computer program includes a mobile application provided to a mobile digital processing device. In some embodiments, the mobile application is provided to a mobile digital processing device at the time it is manufactured. In other embodiments, the mobile application is provided to a mobile digital processing device via the computer network described herein.

In view of the disclosure provided herein, a mobile application is created by techniques known to those of skill in the art using hardware, languages, and development environments known to the art. Those of skill in the art will recognize that mobile applications are written in several languages. Suitable programming languages include, by way of non-limiting examples, C, C++, C#, Objective-C, Java™, Javascript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Android™ Market, BlackBerry® App World, App Store for Palm devices, App Catalog for webOS, Windows® Marketplace for Mobile, Ovi Store for Nokia® devices, Samsung® Apps, and Nintendo® DSi Shop.

In some embodiments, the system disclosed herein includes software, server, and/or database modules, or use of the same. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on cloud computing platforms. In some embodiments, software modules are hosted on one or more machines in one location. In other embodiments, software modules are hosted on one or more machines in more than one location.

In some embodiments, the system disclosed herein includes one or more databases, or use of the same. In view of the disclosure provided herein, those of skill in the art will recognize that many databases are suitable for storage and retrieval of information as described herein. In various embodiments, suitable databases include, by way of non-limiting examples, relational databases, non-relational databases, object oriented databases, object databases, entity-relationship model databases, associative databases, and XML databases. In some embodiments, a database is internet-based. In further embodiments, a database is web-based. In still further embodiments, a database is cloud computing-based. In other embodiments, a database is based on one or more local computer storage devices.

In the above description, an embodiment is an example or implementation of the inventions. The various appearances of “one embodiment,” “an embodiment” or “some embodiments” do not necessarily all refer to the same embodiments.

Although various features of the invention may be described in the context of a single embodiment, the features may also be provided separately or in any suitable combination. Conversely, although the invention may be described herein in the context of separate embodiments for clarity, the invention may also be implemented in a single embodiment.

Reference in the specification to “some embodiments”, “an embodiment”, “one embodiment” or “other embodiments” means that a particular feature, structure, or characteristic described in connection with the embodiments is included in at least some embodiments, but not necessarily all embodiments, of the inventions.

It is to be understood that the phraseology and terminology employed herein is not to be construed as limiting and are for descriptive purpose only.

The principles and uses of the teachings of the present invention may be better understood with reference to the accompanying description, figures and examples.

It is to be understood that the details set forth herein do not construe a limitation to an application of the invention.

Furthermore, it is to be understood that the invention can be carried out or practiced in various ways and that the invention can be implemented in embodiments other than the ones outlined in the description above.

It is to be understood that the terms “including”, “comprising”, “consisting” and grammatical variants thereof do not preclude the addition of one or more components, features, steps, or integers or groups thereof and that the terms are to be construed as specifying components, features, steps or integers.

If the specification or claims refer to “an additional” element, that does not preclude there being more than one of the additional element.

It is to be understood that where the claims or specification refer to “a” or “an” element, such reference is not be construed that there is only one of that element. It is to be understood that where the specification states that a component, feature, structure, or characteristic “may”, “might”, “can” or “could” be included, that particular component, feature, structure, or characteristic is not required to be included. Where applicable, although state diagrams, flow diagrams or both may be used to describe embodiments, the invention is not limited to those diagrams or to the corresponding descriptions. For example, flow need not move through each illustrated box or state, or in exactly the same order as illustrated and described. Methods of the present invention may be implemented by performing or completing manually, automatically, or a combination thereof, selected steps or tasks.

The descriptions, examples, methods and materials presented in the claims and the specification are not to be construed as limiting but rather as illustrative only. Meanings of technical and scientific terms used herein are to be commonly understood as by one of ordinary skill in the art to which the invention belongs, unless otherwise defined. The present invention may be implemented in the testing or practice with methods and materials equivalent or similar to those described herein.

While the invention has been described with respect to a limited number of embodiments, these should not be construed as limitations on the scope of the invention, but rather as exemplifications of some of the preferred embodiments. Other possible variations, modifications, and applications are also within the scope of the invention. Accordingly, the scope of the invention should not be limited by what has thus far been described, but by the appended claims and their legal equivalents.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A device for collecting and characterizing substances in a toilet and cleaning the toilet, the device comprises:
a storage and control module, comprising:
at least one container, said at least one container is configured to store one or more cleaning materials of a first type for cleaning or sanitizing said toilet;
one or more pumps for moving via one or more pipes said one or more cleaning materials of the first type;
at least one diagnostic module configured to receive said substances and diagnose the received substances, the diagnostic module comprises:
a housing having a cavity therein wherein the cavity is configured to contain said substances;
one or more openings for receiving said substances via other one or more pipes;
an imaging module comprising one or more imagers for capturing one or more images of the substances;
an illumination module comprising one or more illumination sources for illuminating the substances;
a sensor module comprising one or more sensors for obtaining sensory data of the substances;
at least one processor in communication with said sensor module and imaging module, said at least one processor is configured to receive said sensory data and said captured images and analyze said sensory data and said captured images to identify the characteristics of said substances; and
one or more power sources;
a treatment module, said treatment module comprising:
a housing having a cavity configured and enabled to collect and store flushing tank fluids and said substances of said toilet flowing via one or more openings in said housing;
one or more cleaning materials of a second type;
a spraying module, comprising one or more spraying devices, wherein said spraying devices are configured and enabled to spray said flushing tank fluids mixed with the cleaning materials of the second type via the one or more spraying devices into to the interior of said toilet bowl;
one or more pumps for drawing said flushing tank fluids mixed within the treatment module with the second type of cleaning materials to the spraying module;
a rotatable spraying module configured to:
receive cleaning materials of the first type from said one or more pipes;
rotate perpendicularly in respect to said treatment module length at the said toilet bowl cavity; and
spray said cleaning materials of the first type at said toilet bowl cavity;
a bridge module connectable to said treatment module and said treatment module, said bridge module is configured and enabled to be hanged on said toilet bowl rim and hold said treatment module away and below the toilet bowl rim at the path of the toilet flushing water.

2. The device of claim 1 comprising a mobile communication device comprising said processor and wireless communication circuitry to couple to the device and communicate with a remote server, the processor comprising instructions to transmit the captured images and the sensory data of the substances to the remote server and receive substances data in response to the captured images and the sensory data from the remote server.

3. The device of claim 1, wherein the remote server comprises a database and a tangible medium embodying instructions of an algorithm to compare the captured images and sensory data to the database.

4. The device of claim 1, wherein the substance data comprises one or more of an identification of the substances, a classification of the substances among a plurality of classifications, one or more components of the substances, or food categories of the substances.

5. The device of claim 1 wherein the treatment module comprises a collecting module attached externally to the treatment module, said collecting module is configured to collect said substances and insert them into the treatment module.

6. The device of claim 5 wherein said collecting module comprises one or more of:
mechanical arms; tubules for collecting and sucking the substances; whip type element configured to perform a whiplash movement and stick the substances within the toilet to its end.

7. The device of claim 1 comprising an optical fiber comprising one or more imagers attached to said optical fiber -distal end said one or more imagers are configured to capture one or more images of the substances and transmit the one or more images to the said device and/or to an external device.

8. A device for characterizing substances in a scene, the device comprising:
a housing having a cavity therein wherein the cavity is configured to contain said substances;
one or more openings for receiving said substances from the scene; an imaging module comprising one or more imagers or video imagers of high resolution for capturing one or more images of the substances;
an illumination module comprising one or more illumination sources for illuminating the substances;
a sensor module comprising one or more sensors for obtaining sensory data of the substances;
a gas supply device for storing and inserting gas into the housing;
a mechanical module configured to accordingly insert or take out the substances into or from the device via the one or more openings;
a syringe configured to add one or more materials into the housing;
a pump for creating a vacuum in the device;
an air pump configured to suck air from the housing and take out the substances from the housing;
a fan for cooling the housing;
a fluid injector for inserting fluid into the housing;
one or more opening for inserting working tools to sterilely move or take the substances or control other modules in the device;
at least one processor in communication with said sensor module and imaging module, said at least one processor is configured to:
receive said sensory data and said captured images;
compare the captured images and the sensory data to a database to identify the characteristics of said substances.

9-49. (canceled)
